SEQUENCE LISTING (2 sequences)

SEQ ID NO: 1 - protein, 31 aa, Homo sapiens
HAEGTFTSDVSSYLEGQAAKEFIAWLVKGRG

SEQ ID NO: 2 - protein, 40 aa, Artificial Sequence
  Exendin-4
HGEGTFITSDLSKQMEEEAVRLFIEWLKNGGPSSGAPPPS

(19) United States
(12) Patent Application Publication
(10) Pub. No.: US 2015/0273069 A1
(43) Pub. Date: Oct. 1, 2015

(54) FATTY ACID ACYLATED AMINO ACIDS FOR ORAL PEPTIDE DELIVERY

(71) Applicant: NOVO NORDISK A/S, Bagsværd (DK)

(72) Inventors: Simon Bjerregaard, Hilleroed (DK); Florian Foeger, Malmoe (SE); Stephen Buckley, Virum (DK); Frantisek Hubalek, Herlev (DK)

(21) Appl. No.: 14/436,126

(22) PCT Filed: Oct. 16, 2013
(86) PCT No.: PCT/EP2013/071618

§ 371 Date: Apr. 16, 2015

Related U.S. Application Data
(60) Provisional application No. 61/715,416, filed on Oct. 18, 2012.

(30) Foreign Application Priority Data
Oct. 17, 2012 (EP) 12188872.1

(51) Int. Cl. A61K 47/18; A61K 9/00; A61K 47/22; A61K 38/26

(52) U.S. Cl. CPC A61K 47/183; A61K 38/26; A61K 9/0053; A61K 47/22

(57) ABSTRACT

The present invention relates to oral pharmaceutical compositions comprising a GLP-1 peptide and a fatty acid acylated amino acid and use thereof.

This invention relates to compositions comprising a GLP-1 peptide and a fatty acid acylated amino acid (FA-aa) as well as use thereof, including use thereof in medicine.

### BACKGROUND

Current GLP-1 therapies are based on invasive and inconvenient parenteral administration. The oral route of administration is non-invasive and has a great potential to decrease the patient's discomfort related to drug administration and to increase drug compliance. However several barriers exist; such as the enzymatic degradation in the gastrointestinal (GI) tract, drug efflux pumps, insufficient and variable absorption from the intestinal mucosa, as well as first pass metabolism in the liver. Until now no products for oral delivery of GLP-1 peptides have been marketed. The oral route of administration is complex and establishment of a composition suitable for treatment of patients with an effective bioavailability of GLP-1 is desired.

### SUMMARY

In some embodiments the invention relates to an oral pharmaceutical composition comprising a GLP-1 peptide and at least one amino acid acylated at a free amino group with a fatty acid comprising an alkyl group consisting of 5 to 19 carbon atoms.

In some embodiments the invention relates to the composition as defined herein for use as a medicament. In some embodiments the invention relates to the composition as defined herein for the treatment and/or prevention of diabetes.

In some embodiments the invention relates to use of the composition as defined herein for increasing the oral bioavailability of a GLP-1 peptide. In some embodiments the invention relates to a method for increasing bioavailability of a GLP-1 peptide comprising oral administration of a composition as defined herein to a subject.

### BRIEF DESCRIPTION OF DRAWINGS

FIG. 1 shows plasma exposure of semaglutide in rats following gut injection of 100 μl of aqueous formulations of 1000 nmol/ml semaglutide and one of 55 mg/ml sodium N-decanoyl L-leucinate (squares), 55 mg/ml sodium N-cocoyl L-glutamate (diamonds), or 55 mg/ml N-cocoyl glycinate (triangles); results are shown as mean±SEM, n=6.

FIG. 2 shows plasma exposure of semaglutide in rats following gut injection of 100 μl of injection of aqueous formulations of 1000 nmol/ml semaglutide and one of 55 mg/ml sodium N-myristoyl L-glutamate (squares) or 55 mg/ml sodium N-dodecanoyl L-glutamate (triangles); results are shown as mean±SEM, n=6.

FIG. 3 shows plasma exposure of semaglutide in rats following gut injection of 100 μl of an aqueous formulation of 1000 nmol/ml semaglutide and 55 mg/ml sodium N-dodecanoyl L-sarcosinate (squares); results are shown as mean±SEM, n=6.

FIG. 4 shows plasma exposure of semaglutide following administration of a solid dosage form containing 23 mg sodium N-myristoyl L-glutamate and 100 nmol semaglutide; results are shown as mean±SEM, n=8.

### DESCRIPTION

The present invention relates to pharmaceutical compositions comprising a GLP-1 peptide and a fatty acid acylated amino acid (FA-aa). The FA-aa's of the invention were surprisingly found to be permeation enhancers suitable for oral administration of GLP-1 peptides. In some embodiments the term “permeation enhancer” when used herein refers to compounds that promote the absorption of the GLP-1 peptide across the gastrointestinal tract. In some embodiments the FA-aa of the invention is suitable for increasing the bioavailability and/or absorption of GLP-1 peptides. A FA-aa is an amino acid-based surfactant and thus a mild and biodegradable surfactant with low toxicity. In some embodiments the term “surfactant” refers to any substance, in particular a detergent, that can adsorb at surfaces and interfaces, such as but not limited to liquid to air, liquid to liquid, liquid to container or liquid to any solid. In some embodiments the surfactant has no charged groups in its hydrophilic groups. In some embodiments it was surprisingly found that the fatty acid N-acylated amino acids of the invention increase the absorption of GLP-1 peptides after oral administration to a higher degree than commonly used permeation enhancers known in the art.

Due to their low toxicity and increasing effect on oral bioavailability of GLP-1 peptides the FA-aa according to the present invention are valuable ingredients in oral pharmaceutical compositions. The term “oral bioavailability” as used herein refers to the the amount of administered drug and/or active moieties thereof in systemic circulation after oral administration (estimated as the area under the plasma concentration versus time curve) relative to the amount of administered drug and/or active moieties thereof in systemic circulation after intravenous administration of said drug.

In some embodiments the invention relates to use of the pharmaceutical composition as defined herein for increasing the oral bioavailability of a GLP-1 peptide.

In some embodiments the invention relates to a method for increasing bioavailability of a GLP-1 peptide comprising oral administration of the pharmaceutical composition as defined herein.

In some embodiments the invention relates to a method for increasing bioavailability of a GLP-1 peptide comprising the steps of including a FA-aa in a pharmaceutical composition of a GLP-1 peptide administered to a subject.

In some embodiments the invention relates to a method for increasing the plasma concentration of a GLP-1 peptide comprising the step of exposing the gastrointestinal tract of a subject to a pharmaceutical composition comprising a GLP-1 peptide and a FA-aa resulting in an increased plasma concentration of said GLP-1 peptide in said subject. In some embodiments said exposure is achieved by oral administration of said pharmaceutical composition.

In some embodiments the invention relates to a method for increasing the uptake of a GLP-1 peptide comprising the step of: exposing the gastrointestinal tract of a subject to a GLP-1 peptide and at least one FA-aa, whereby the plasma concentration of said GLP-1 peptide in said subject is increased compared to an exposure not including the at least one FA-aa.

In some embodiments the invention relates to a method for increasing uptake of a GLP-1 peptide across an/the epithelia cell layer of the gastro intestinal tract comprising the steps of administering a pharmaceutical composition comprising a GLP-1 peptide and at least one FA-aa to a subject, whereby an increased uptake of said GLP-1 peptide is obtained compared to the uptake of said GLP-1 peptide obtained when said GLP-1 peptide composition does not include the at least one FA-aa.

### Fatty Acid Acylated Amino Acid

The invention relates to a pharmaceutical composition comprising i) a GLP-1 peptide and ii) at least one fatty acid amino acid (FA-aa) or a salt of said FA-aa. In some embodiments the pharmaceutical composition is an oral pharmaceutical composition. In some embodiments the pharmaceutical composition comprising a GLP-1 peptide and at least one amino acid acylated at a free amino group with a fatty acid, which are referred to as fatty acid acylated amino acids (FA-aa) herein. In some embodiments the invention relates to a pharmaceutical composition comprising a GLP-1 peptide and at least one amino acid acylated at its alpha-amino group with a fatty acid. In some embodiments the term “amino acid” as used herein refers to any molecule that contains both amine and carboxyl functional groups.

In some embodiments the FA-aa comprises the amino acid residue of a non-cationic amino acid. In some embodiments the FA-aa may be represented by the general formula A-X, wherein A is the amino acid residue of a non-cationic amino acid and X is a fatty acid attached by acylation to A's alpha-amino group. In some embodiments the term “non-cationic amino acid” refers to an amino acid selected from the group consisting of non-polar hydrophobic amino acids, polar non-charged amino acids, and polar acidic amino acids. In some embodiments the term “non-cationic amino acid” as used herein refers to amino acids selected from the group consisting of Alanine (Ala), Valine (Val), Leucine (Leu), Isoleucine (Ile), Phenylalanine (Phe), Tryptophane (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp), and Glutamic acid (Glu).

In some embodiments the FA-aa comprises the amino acid residue of a non-polar hydrophobic amino acid. In some embodiments the FA-aa may be represented by the general formula A-X, wherein A is the amino acid residue of a non-polar hydrophobic amino acid and X is a fatty acid attached by acylation to A's alpha-amino group. In some embodiments the term “non-polar hydrophobic amino acid” as used herein refers to categorisation of amino acids used by the person skilled in the art. In some embodiments the term “non-polar hydrophobic amino acid” refers to an amino acid selected from the group consisting of Alanine (Ala), Valine (Val), Leucine (Leu), Isoleucine (Ile), Phenylalanine (Phe), Tryptophane (Trp), Methionine (Met), Proline (Pro) and Sarcosine.

In some embodiments the FA-aa comprises the amino acid residue of a polar non-charged amino acid. In some embodiments the FA-aa may be represented by the general formula A-X, wherein A is the amino acid residue of a polar non-charged amino acid and X is a fatty acid attached by acylation to A's alpha-amino group. In some embodiments the term “polar non-charged amino acid” as used herein refers to categorisation of amino acids used by the person skilled in the art. In some embodiments the term “polar non-charged amino acid” refers to an amino acid selected from the group consisting of Glycine (Gly), Serine (Ser), Threonine (Thr), Cysteine (Cys), Tyrosine (Tyr), Asparagine (Asn), and Glutamine (Gin).

In some embodiments the FA-aa comprises the amino acid residue of a polar acidic amino acid. In some embodiments the FA-aa may be represented by the general formula A-X, wherein A is the amino acid residue of a polar acidic amino acid and X is a fatty acid attached by acylation to A's alpha-amino group. In some embodiments the term “polar acidic amino acid” as used herein refers to categorisation of amino acids used by the person skilled in the art. In some embodiments the term “polar acidic amino acid” refers to an amino acid selected from the group consisting of Aspartic acid (Asp) and Glutamic acid (Glu).

In some embodiments the amino acid residue of the FA-aa comprises the amino acid residue of an amino acid that is not encoded by the genetic code. Modifications of amino acids by acylation may be readily performed using acylation agents known in the art that react with the free alpha-amino group of the amino acid.

In some embodiments amino acids or the amino acid residues herein are in the L-form unless otherwise stated.

In some embodiments the amino acid residue is in the free acid form and/or a salt thereof, such as a sodium (Na+) salt thereof.

The FA-aa may be represented by the general formula I:

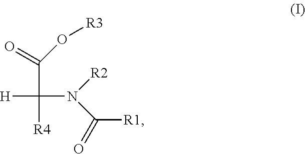

wherein R1 is an alkyl group comprising 5 to 19 carbon atoms; R2 is H (i.e. hydrogen), CH₃ (i.e. methyl group), or covalently attached to R4 via a (CH₂)₃ group; R3 is H or absent; and R4 is an amino acid side chain or covalently attached to R2 via a (CH₂)₃ group; or a salt thereof.

The FA-aa of the invention is acylated with a fatty acid comprising an alkyl group consisting of 5 to 19 carbon atoms. In some embodiments the alkyl group consists of 7 to 17 carbon atoms. In some embodiments the alkyl group consists of 9-15 carbon atoms. In some embodiments the alkyl group consists of 11-13 carbon atoms. In some embodiments the alkyl group consists of 9 carbon atoms. In some embodiments the alkyl group consists of 11 carbon atoms. In some embodiments the alkyl group consists of 13 carbon atoms. In some embodiments the alkyl group consists of 15 carbon atoms. In some embodiments the alkyl group consists of 17 carbon atoms.

The nomenclature as used herein for an FA-aa of the invention first denotes the fatty acid acylation group, such as e.g. dodecanoyl for CH₃(CH₂)₁₀C(O)—, followed by the amino acid which is being acylated on its alpha-amino group such as e.g. L-alanine. For example, the FA-aa named “N-dodecanoyl-L-alanine” is the same as the FA-aa of general formula I

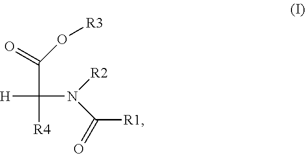

wherein R1 is an alkyl group consisting of 11 carbon atoms, R2 is H, R3 is H, R4 is the amino acid side chain of alanine and thus CH₃ (ie. a methyl group) and the configuration is of alanine is L.

In the instance where the FA-aa forms a salt e.g. with an alkali metal, the naming starts with denoting the alkali metal forming the salt such as e.g. sodium, followed by the fatty acid acylation group and ending by the amino acid which is being acylated on its alpha-amino group. For example, the FA-aa named “sodium N-dodecanoyl alaninate” is the same as the FA-aa of general formula I

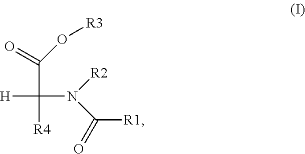

wherein R1 is an alkyl group consisting of 11 carbon atoms, R2 is H, R3 is absent, R4 is the amino acid side chain of alanine and thus CH₃ (ie. a methyl group) and sodium forms a salt with said FA-aa.

In some embodiments the FA-aa is soluble at intestinal pH values, particularly in the range pH 5.5 to 8.0, such as in the range pH 6.5 to 7.0. In some embodiments the FA-aa is soluble below pH 9.0.

In some embodiments the FA-aa has a solubility of at least 5 mg/mL. In some embodiments the FA-aa has a solubility of at least 10 mg/mL. In some embodiments the FA-aa has a solubility of at least 20 mg/mL. In some embodiments the FA-aa has a solubility of at least 30 mg/mL. In some embodiments the FA-aa has a solubility of at least 40 mg/mL. In some embodiments the FA-aa has a solubility of at least 50 mg/mL. In some embodiments the FA-aa has a solubility of at least 60 mg/mL. In some embodiments the FA-aa has a solubility of at least 70 mg/mL. In some embodiments the FA-aa has a solubility of at least 80 mg/mL. In some embodiments the FA-aa has a solubility of at least 90 mg/mL. In some embodiments the FA-aa has a solubility of at least 100 mg/mL. In some embodiments solubility of the FA-aa is determined in an aqueous solution at a pH value 1 unit above or below pKa of the FA-aa at 37° C. In some embodiments solubility of the FA-aa is determined in an aqueous solution at pH 8 at 37° C. In some embodiments solubility of the FA-aa is determined in an aqueous solution at a pH value 1 unit above or below pl of the FA-aa at 37° C. In some embodiments solubility of the FA-aa is determined in an aqueous solution at a pH value 1 units above or below pl of the FA-aa at 37° C., wherein said FA-aa two or more ionisable groups with opposite charges. In some embodiments solubility of the FA-aa is determined in an aqueous 50 mM sodium phosphate buffer, pH 8.0 at 37° C.

In some embodiments the FA-aa is selected from the group consisting of formula (a), (b), (c), (d), (e), (f), (g), (h), (i), (j), (k), (l), (m), (n), (o), (p), (q), and (r), wherein R1 is an alkyl group comprising 5 to 19 carbon atoms, R2 is H (i.e. hydrogen) or CH₃(i.e. methyl group), and R3 is H; or a salt or the free acid form thereof.

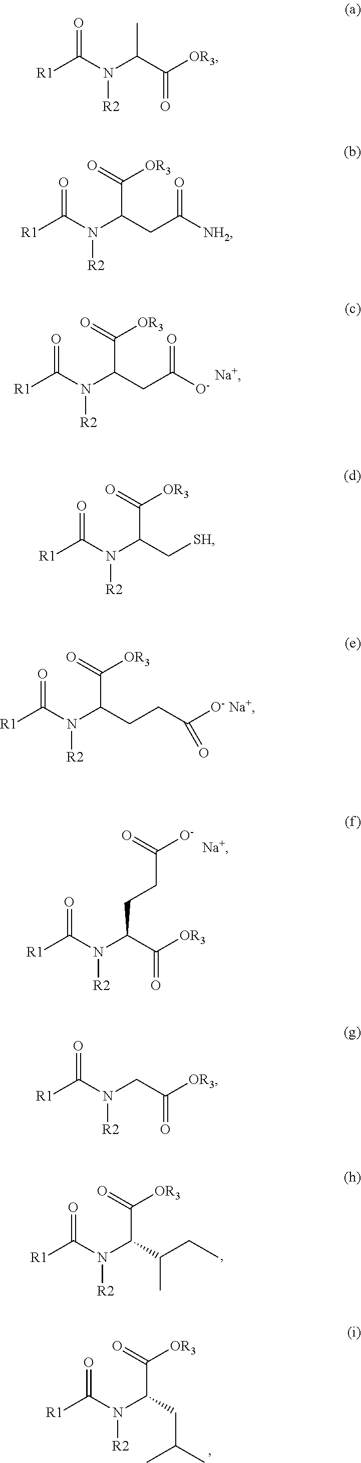

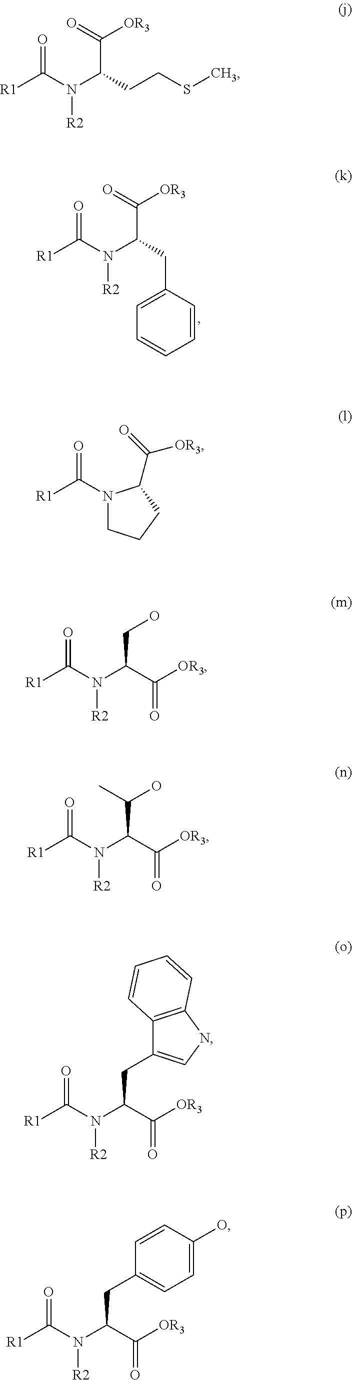

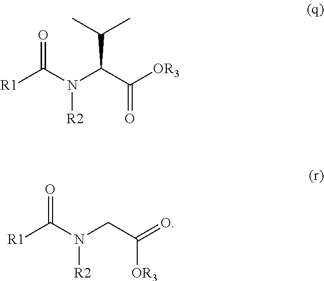

In some embodiments the FA-aa is selected from the group consisting of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophane, sodium N-dodecanoyl valinate, N-dodecanoyl-L-valine, sodium N-dodecanoyl sarcosinate, N-dodecanoyl-L-sarcosine, sodium N-dodecanoyl sarcosinate, sodium N-oleoyl sarcosinate and sodium N-decyl leucine.

In some embodiments the FA-aa is selected from the group consisting of sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decyl leucine. In some embodiments the FA-aa is selected from the group consisting of sodium N-decanoyl alaninate, N-decanoyl-L-alanine, sodium N-decanoyl isoleucinate, N-decanoyl-L-isoleucine, sodium N-decanoyl leucinate, N-decanoyl-L-leucine, sodium N-decanoyl methioninate, N-decanoyl-L-methionine, sodium N-decanoyl phenylalaninate, N-decanoyl-L-phenylalanine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophane, sodium N-decanoyl valinate, N-decanoyl-L-valine, sodium N-decanoyl sarcosinate, and N-decanoyl-L-Sarcosine.

In some embodiments the FA-aa may be selected from the group consisting of sodium N-dodecanoyl alaninate, N-dodecanoyl-L-alanine, sodium N-dodecanoyl leucinate, N-dodecanoyl-L-leucine, sodium N-dodecanoyl phenylalaninate, N-dodecanoyl-L-phenylalanine, sodium N-dodecanoyl valinate, and N-dodecanoyl-L-valine.

In some embodiments the FA-aa may be selected from the group consisting of sodium N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl threoninate, N-decanoyl-L-threonine, sodium N-decanoyl tyrosinate and N-decanoyl-L-tyrosine, sodium N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl threoninate, N-decanoyl-L-threonine, sodium N-decanoyl tyrosinate, and N-decanoyl-L-tyrosine.

In some embodiments the FA-aa may be selected from the group consisting of sodium N-dodecanoyl asparaginate, N-dodecanoyl-L-asparagine, sodium N-dodecanoyl aspartic acid, N-dodecanoyl-L-aspartic acid, sodium N-dodecanoyl glutamic acid, N-dodecanoyl-L-glutamic acid, sodium N-decanoyl asparaginate, N-decanoyl-L-asparagine, sodium N-decanoyl aspartic acid, N-decanoyl-L-aspartic acid, sodium N-decanoyl glutamic acid and N-decanoyl-L-glutamic acid.

In some embodiments the FA-aa may be selected from the group consisting of Amisoft HS-11 P (sodium Stearoyl Glutamate, Amisoft MS-11 (sodium Myristoyl Glutamate)), Amisoft LS-11 (sodium Dodecanoyl Glutamate), Amisoft CS-11 (sodium Cocoyl Glutamate) and sodium N-cocoyl glutamate, sodium N-dodecanoyl asparaginate, N-dodecanoyl-L-asparagine, sodium N-dodecanoyl aspartic acid, N-dodecanoyl-L-aspartic acid, sodium N-dodecanoyl glutamic acid, N-dodecanoyl-L-glutamic acid, sodium N-decanoyl asparaginate, N-decanoyl-L-asparagine, sodium N-decanoyl aspartic acid, N-decanoyl-L-aspartic acid, sodium N-decanoyl glutamic acid, and N-decanoyl-L-glutamic acid.

In some embodiments the FA-aa may be selected from the group consisting of Amisoft HS-11 P (sodium Stearoyl Glutamate, Amisoft MS-11 (sodium Myristoyl Glutamate)), Amisoft LS-11 (sodium Dodecanoyl Glutamate), Amisoft CS-11 (sodium Cocoyl Glutamate), and sodium N-cocoyl glutamate.

In some embodiments the FA-aa may be selected from the group consisting of sodium N-dodecanoyl asparaginate, N-dodecanoyl-L-asparagine, sodium N-dodecanoyl aspartic acid, N-dodecanoyl-L-aspartic acid, sodium N-dodecanoyl glutamic acid, N-dodecanoyl-L-glutamic acid, sodium N-decanoyl asparaginate, N-decanoyl-L-asparagine, sodium N-decanoyl aspartic acid, N-decanoyl-L-aspartic acid, sodium N-decanoyl glutamic acid, and N-decanoyl-L-glutamic acid.

In some embodiments the FA-aa may be selected from the group consisting of Amisoft HS-11 P (sodium N-stearoyl glutamate), Amisoft MS-11 (sodium N-myristoyl glutamate)), Amisoft LS-11 (sodium N-dodecanoyl glutamate), Amisoft CS-11 (sodium N-cocoyl glutamate), and sodium N-cocoyl glutamate.

In some embodiments the FA-aa may be selected from the group consisting of sodium N-dodecanoyl asparaginate, N-dodecanoyl-L-asparagine, sodium N-dodecanoyl aspartic acid, N-dodecanoyl-L-aspartic acid, sodium N-dodecanoyl glutamic acid, N-dodecanoyl-L-glutamic acid, sodium N-decanoyl asparaginate, N-decanoyl-L-asparagine, sodium N-decanoyl aspartic acid, N-decanoyl-L-aspartic acid, sodium N-decanoyl glutamic acid, and N-decanoyl-L-glutamic acid.

In some embodiments the FA-aa may be selected from the group consisting of Amisoft HS-11 P (sodium Stearoyl Glutamate, Amisoft MS-11 (sodium Myristoyl Glutamate)), Amisoft LS-11 (sodium Dodecanoyl Glutamate), Amisoft CS-11 (sodium Cocoyl Glutamate) and sodium N-cocoyl glutamate.

The following FA-aa's are commercially available:

| | | Provider |
|---|---|---|
| Brand name | Chemical name | (per 14-APR-2011) |
| Hamposyl L-95 | sodium N-dodecanoyl | Chattem Chemicals |
| | sarcosinate | |
| Hamposyl O | sodium N-oleoyl | Chattem Chemicals |
| | sarcosinate | |
| Hamposyl C | sodium N-cocoyl | Chattem Chemicals |
| | sarcosinate | |
| Hamposyl L-30 | sodium N-dodecanoyl | Chattem Chemicals |
| | sarcosinate | |
| Amisoft HS-11 P | sodium N-stearoyl | Ajinomoto |
| | glutamate | |
| Amisoft LS-11 | sodium N-dodecanoyl | Ajinomoto |
| | glutamate | |
| Amisoft CS-11 | sodium N-cocoyl glutamate | Ajinomoto |
| Amisoft MS-11 | sodium N-myristoyl | Ajinomoto |
| | glutamate | |
| Amilite GCS-11 | sodium N-cocoyl glycinate | Ajinomoto |

In some embodiments the terms “fatty acid N-acylated amino acid”, “fatty acid acylated amino acid”, or “acylated amino acid” are interchangeably herein and refer to an amino acid that is acylated with a fatty acid at its alpha-amino group.

### Pharmaceutical Compositions

The FA-aa of the invention may be part of a pharmaceutical composition. The pharmaceutical composition may be an oral pharmaceutical composition. In some embodiments the composition comprises a GLP-1 peptide and at least one FA-aa. The terms “composition” or “pharmaceutical composition” are used interchangeably herein and refer to a pharmaceutical composition. In some embodiments the composition comprises at least one GLP-1 peptide and at least one FA-aa. In some embodiments the composition comprises at least one GLP-1 peptide and two or more FA-aa's (i.e. different FA-aa's). In some embodiments the composition comprises one or more commercially available FA-aa's.

In some embodiments the composition comprises at least one pharmaceutically acceptable excipient. The term “excipient” as used herein broadly refers to any component other than the active therapeutic ingredient(s). The excipient may be an inert substance, which is inert in the sense that it substantially does not have any therapeutic and/or prophylactic effect per se. The excipient may serve various purposes, e.g. as a delivery agent, absorption enhancer, vehicle, filler (also known as diluents), binder, lubricant, glidant, disintegrant, crystallization retarders, acidifying agent, alkalizing agent, preservative, antioxidant, buffering agent, chelating agent, complexing agents, surfactant agent, emulsifying and/or solubilizing agents, sweetening agents, wetting agents stabilizing agent, colouring agent, flavouring agent, and/or to improve administration, and/or absorption of the active substance. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In some embodiments the composition comprises a preservative, such as phenol, m-cresol, or a mixture of phenol and m-cresol. The term “preservative” as used herein refers to a compound which is added to a composition to prevent or delay microbial activity (growth and metabolism).

The components of the composition may be present in any relative amounts. In some embodiments the composition comprises up to 90% surfactant; or up to 90% polar organic solvent, such as polyethylene glycol (PEG) 300 g/mol, PEG 400 g/mol, PEG 600 g/mol, PEG 1000 g/mol; or up to 90% of a lipid component. PEGs are prepared by polymerization of ethylene oxide and are commercially available over a wide range of molecular weights from 300 g/mol to Ser. No. 10/000,000 g/mol.

The composition may be liquid (e.g. aqueous), semisolid, or solid. In some embodiments the composition is in the form of a solid, such as capsules, tablets, dragees, pills, lozenges, powders, and granules. In some embodiments the term “solid” refers to liquid compositions encapsulated in a soft or hard capsule technology in addition to other solid compositions, such as tablets and multiparticulates. Multiparticulates may be pellets, microparticles, nanoparticles, liquid or semisolid fill formulations in soft or hard capsules, or enteric coated soft or hard capsules. In some embodiments the composition is in the form of a liquid or semisolid.

In some embodiments the composition comprises at least one pH neutralised GLP-1 peptide. In some embodiments the pH neutralised GLP-1 peptide is prepared by dissolving the GLP-1 peptide and adjusting the pH of the resulting solution to a value, which is 1 unit, alternatively 2 units and alternatively 2.5 pH units, above or below the pl of the GLP-1 peptide, where after said resulting solution is optionally freeze or spray dried, said pH adjustment may be performed with a non-volatile acid or base.

### SEDDS, SMEDDS or SNEDDS

In some embodiments the composition is a SEDDS, SMEDDS or SNEDDS. The SEDDS, SMEDDS or SNEDDS may be solid, liquid or semisolid. In some embodiments SEDDS, SMEDDS or SNEDDS may be seen as pre-concentrates because they spontaneously form colloidal structures, such as emulsions, microemulsions, nanoemulsions, and/or other colloidal systems, e.g., oil-in-water emulsion, oil-in-water microemulsion or oil-in-water nanoemulsion, swollen micelle, micellar solution, when the SEDDS, SMEDDS or SNEDDS is exposed to an aqueous medium under conditions of gentle agitation (e.g. by simple shaking by hand for a short period of time, for example for ten seconds) or when the composition is exposed to the gastrointestinal fluids after oral administration and the digestive motility that would be encountered in the GI tract. “SEDDS” (self-emulsifying drug delivery systems) are herein defined as mixtures of a hydrophilic component, a surfactant, optionally a co-surfactant or lipid component, and a GLP-1 peptide that spontaneously forms an oil-in-water emulsion when exposed to aqueous media under conditions of gentle agitation or digestive motility that would be encountered in the GI tract. “SMEDDS” (self-micro-emulsifying drug delivery systems) are herein defined as isotropic mixtures of a hydrophilic component, a surfactant, optionally a co-surfactant or lipid component, and a GLP-1 peptide that rapidly form an oil-in-water microemulsion or nanoemulsion when exposed to aqueous media under conditions of gentle agitation or digestive motility that would be encountered in the GI tract. “SNEDDS” (self-nano-emulsifying drug delivery systems) are herein defined as isotropic mixtures of a hydrophilic component, at least one surfactant with HLB above 10, optionally a co-surfactant, optionally a lipid component, and a GLP-1 peptide that rapidly form a nanoemulsion (droplet size below 20 nm in diameter as e.g. measured by PCS) when exposed to aqueous media under conditions of gentle agitation or digestive motility that would be encountered in the GI tract. In some embodiments the term “emulsion” refers to a slightly opaque, opalescent or opaque colloidal coarse dispersion that is formed spontaneously or substantially spontaneously when its components are brought into contact with an aqueous medium. In some embodiments an emulsion contains homogenously dispersed particles or domains, for example of a solid or liquid state (e.g., liquid lipid particles or droplets), of a mean diameter of more than 150 nm as measured by standard light scattering techniques, e.g. using a MALVERN ZETASIZER Nano ZS. In some embodiments the term “microemulsion” refers to a clear or translucent, slightly opaque, opalescent, non-opaque or substantially non-opaque colloidal dispersion that is formed spontaneously or substantially spontaneously when its components are brought into contact with an aqueous medium; a microemulsion is thermodynamically stable and contains homogenously dispersed particles or domains, for example of a solid or liquid state (e.g., liquid lipid particles or droplets), of a mean diameter of less than 150 nm as measured by standard light scattering techniques, e.g. using a MALVERN ZETASIZER Nano ZS. In some embodiments when the composition is brought into contact with an aqueous medium a microemulsion is formed which contains homogenously dispersed particles or domains of a mean diameter of less than 100 nm, such as less than 50 nm, less than 40 nm and less than 30 nm. In some embodiments “domain” refers to an area of a composition with predominantly lipophilic or hydrophilic composition and said domain may be spherical or have other shapes, such as rod-like or oval. As used herein, the term “nanoemulsion” refers to a clear or translucent, slightly opaque, opalescent, non-opaque or substantially non-opaque colloidal dispersion with particle or droplet size below 20 nm in diameter (as e.g. measured by PCS) that is formed spontaneously or substantially spontaneously when its components are brought into contact with an aqueous medium. In some embodiments when the composition is brought into contact with an aqueous medium a nanoemulsion is formed which contains homogenously dispersed particles or domains of a mean diameter of less than 20 nm, such as less than 15 nm, less than 10 nm. In some embodiments when the composition is brought into contact with an aqueous medium a nanoemulsion is formed which contains homogenously dispersed particles or domains of a mean diameter of less than 20 nm, such as less than 15 nm, less than 10 nm, and optionally greater than about 2-4 nm. The SEDDS, SMEDDS or SNEDDS self-emulsifies upon dilution in an aqueous medium for example in a dilution of 1:5, 1:10, 1:50, 1:100 or higher. In some embodiments the composition forms the microemuslion or nanoemulsion comprising particles or domains of a size below 100 nm in diameter. In some embodiments the term “domain size”, “particle size” or “droplet size” as used herein refers to repetitive scattering units and may be measured by e.g., small angle X-ray. In some embodiments the domain size, particle size or droplet size is less than 150 nm, such as less than 100 nm or less than 50 nm. In some embodiments the domain size, particle size or droplet size is less than 20 nm, such as less than 15 nm or less than 10 nm.

In some embodiments the composition comprises at least one GLP-1 peptide, at least one FA-aa, propylene glycol, and at least one non-ionic surfactant (such as at least two non-ionic surfactants). In some embodiments the term “non-ionic surfactant” as used herein refers to any substance, in particular a detergent, that can adsorb at surfaces and interfaces, like liquid to air, liquid to liquid, liquid to container or liquid to any solid and which has no charged groups in its hydrophilic group(s) (sometimes referred to as “heads”). The non-ionic surfactant may be selected from a detergent such as ethoxylated castor oil, polyglycolyzed glycerides, acetylated monoglycerides and sorbitan fatty acid esters, polysorbate such as polysorbate-20, polysorbate-40, polysorbate-60, polysorbate-80, super refined polysorbate 20, super refined polysorbate 40, super refined polysorbate 60 and super refined polysorbate 80 (where the term “super refined” is used by the supplier Croda for their high purity Tween products), poloxamers such as poloxamer 188 and poloxamer 407, polyoxyethylene sorbitan fatty acid esters, polyoxyethylene derivatives such as alkylated and alkoxylated derivatives (Tweens, e.g. Tween-20 or Tween-80), block copolymers such as polyethyleneoxide/polypropyleneoxide block copolymers (e.g. Pluronics/Tetronics, Triton X-100 and/or Synperonic PE/L 44 PEL) and ethoxylated sorbitan alkanoates surfactants (e. g. Tween-20, Tween-40, Tween-80, Brij-35), diglycerol laurate, diglycerol caprate, diglycerol caprylate, diglycerol monocaprylate, polyglycerol laurate, polyglycerol caprate, and polyglycerol caprylate.

In some embodiments the composition comprises at least one GLP-1 peptide, at least one FA-aa, and propylene glycol. In some embodiments the composition comprises from 5% to 20% propylene glycol, such as 5% (w/w) to 20% (w/w) propylene glycol.

In some embodiments the composition comprises at least one GLP-1 peptide, at least one FA-aa, propylene glycol, polysorbate 20, and a co-surfactant. In some embodiments polysorbate 20 is a polysorbate surfactant whose stability and relative non-toxicity allows it to be used as a detergent and emulsifier in a number of domestic, scientific, and pharmacological applications; the number 20 refers to the total number of oxyethylene —(CH₂CH₂O)— groups found in the molecule.

In some embodiments the composition comprises at least one GLP-1 peptide, at least one FA-aa, propylene glycol, polysorbate 20, and a polyglycerol fatty acid ester.

In some embodiments, the oral pharmaceutical composition comprises at least one GLP-1 peptide, at least one FA-aa, propylene glycol, polysorbate 20 and a co-surfactant.

In some embodiments, the oral pharmaceutical composition comprises at least one GLP-1 peptide, at least one FA-aa, propylene glycol, polysorbate 20 and a polyglycerol fatty acid ester such as diglycerol monocaprylate.

In some embodiments the composition comprises a polar or semipolar solvent, such as water or propylene glycol.

In some embodiments the composition is a liquid and comprises at least one GLP-1 peptide, at least one FA-aa, at least one polyglycerol fatty acid ester, at least one polyethylene glycol sorbitan fatty acid ester (such as Tween 20 or Tween 85), and optionally a polar or semipolar solvent (such as water or propylene glycol). In some embodiments the sorbitan fatty acid ester is selected from the group consisting of Span 10, Span 20, Span 40, Span 60, or Span 80. In some embodiments the sorbitan fatty acid ester is selected from the group consisting of sorbitan laurate (commercially known as Span 20), sorbitan mono palmitate (commercially known as Span 40), sorbitan mono stearate (commercially known as Span 60), and sorbitan oleate (commercially known as Span 80).

In some embodiments the composition is a liquid and comprises at least one GLP-1 peptide, at least one FA-aa, at least one polyglycerol fatty acid ester, at least one polyethylene glycol sorbitan fatty acid ester, and a polar or semipolar solvent, wherein the composition forms a microemulsion after dilution in an aqueous medium. In some embodiments the polyethylene glycol sorbitan fatty acid ester, which may be selected form the group consisting of Tween 20, Tween 21, Tween 40, Tween 60, Tween 65, Tween 80, Tween 81, and Tween 85. In some embodiments the polyethylene glycol sorbitan fatty acid ester is a polyethylene glycol sorbitan trioleate (commercially known as Tween 85) or polyethylene glycol sorbitan monolaurate (commercially known as Tween 20).

In some embodiments the composition is a liquid and comprises at least one GLP-1 peptide, at least one FA-aa, at least one polyglycerol fatty acid ester, at least one sorbitan fatty acid ester, and optionally a polar or semipolar solvent.

In some embodiments the composition comprises at least one FA-aa, propylene glycol, polysorbate 20, and a co-surfactant. In some embodiments the composition comprises at least one FA-aa, propylene glycol, polysorbate 20, and a polyglycerol fatty acid ester, such as diglycerol monocaprylate. Polysorbate 20 is a polysorbate surfactant whose stability and relative non-toxicity allows it to be used as a detergent and emulsifier in a number of domestic, scientific, and pharmacological applications; the number 20 refers to the total number of oxyethylene —(CH₂CH₂O)— groups found in the molecule.

In some embodiments the composition comprises at least one GLP-1 peptide, at least one FA-aa, at least one high HLB surfactant, at least one low HLB co-surfactant, and a polar solvent. In some embodiments a low HLB surfactant has a HLB value <10 and/or is oil soluble. The term “co-surfactant” as used herein refers to an additional surfactant added to a composition, wherein a first surfactant is present. In some embodiments the composition comprises at least one GLP-1 peptide, at least one FA-aa, at least two high HLB surfactants, and a polar solvent. In some embodiments a high HLB surfactant has a HLB value >10 and/or is water soluble.

### Water Content

In some embodiments the composition comprises less than 10% (w/w) water. In some embodiments the composition comprises less than 9% (w/w) water. In some embodiments the composition comprises less than 8% (w/w) water. In some embodiments the composition comprises less than 7% (w/w) water. In some embodiments the composition comprises less than 6% (w/w) water. In some embodiments the composition comprises less than 5% (w/w) water. In some embodiments the composition comprises less than 4% (w/w) water. In some embodiments the composition comprises less than 3% (w/w) water. In some embodiments the composition comprises less than 2% (w/w) water. In some embodiments the composition comprises less than 1% (w/w) water. In some embodiments the composition comprises 0% (w/w) water.

In some embodiments the composition is non-aqueous. In some embodiments the term “non-aqueous” refers to a composition to which no water is added during preparation of the composition. It is known to the person skilled in the art that a composition which has been prepared without addition of water may take up small amounts of water from the surroundings during handling of the pharmaceutical composition, such as e.g. a soft capsule or a hard capsule used to encapsulate the composition. Also, the GLP-1 peptide and/or one or more of the excipients in the composition may have small amounts of water bound to it before preparing the composition according to the invention. The non-aqueous composition according to the invention may thus contain small amounts of water. In some embodiments the non-aqueous composition comprises less than 10%(w/w) water, such as less than 5%(w/w) water, less than 4%(w/w) water, or less than 3%(w/w) water, or such as less than 2%(w/w) water or less than 1%(w/w) water.

### Encapsulation

The composition of the invention may be encapsulated. The composition (e.g. liquid or semisolid SEDDS, SMEDDS or SNEDDS compositions comprising a GLP-1 peptide and a FA-aa) may be encapsulated with any available soft or hard capsule technology. In some embodiments the soft capsule technology used for encapsulating a composition according to the present invention is gelatine free. In some embodiments the soft capsule technology is the gelatine free Vegicaps® (available from Catalent®). In some embodiments the term “enteric soft or hard capsule technology” when used herein refers to soft or hard capsule technology comprising at least one element with enteric properties, such as at least one layer of an enteric coating.

The composition of the invention may comprise one or more enteric or modified release coatings. The composition may comprise one or more enteric or modified release coatings in addition to soft or hard capsule technology. The enteric or modified release coating may be poly(meth)acrylates, commercially known as Eudragit®. In some embodiments the enteric or modified release coating comprises at least one release modifying polymer which may be used to control the site where the GLP-1 peptide is released. The release modifying polymer may be a polymethacrylate polymer, such as those sold under the Eudragit® trade name (Evonik Rohm GmbH, Darmstadt, Germany), for example Eudragit® L30 D55, Eudragit® L100-55, Eudragit® L100, Eudragit® S100, Eudragit® S12,5, Eudragit® FS30D, Eudragit® NE30D and mixtures thereof as e.g. described in Eudragit® Application Guidelines, Evonik Industries, 11th edition, September 2009. In some embodiments the term “enteric coating” as used herein means a polymer coating that controls disintegration and release of the oral dosage form; the site of disintegration and release of the solid dosage form may be designed depending on the pH of the targeted area, where absorption of the GLP-1 peptide is desired, thus also includes acid resistant protective coatings; the term includes known enteric coatings, but also any other coating with enteric properties, wherein said term “enteric properties” means properties controlling the disintegration and release of the solid oral dosage form (i.e. the oral pharmaceutical composition according to this invention). In some embodiments the term “modified release coating” as used herein refers to a coating which comprises special excipients (e.g. a polymer) or which is prepared by special procedures, or both, designed to modify the rate, the place or the time at which the active substance(s) are released. In some embodiments modified release coating include prolonged-release coating, delayed-release coating, and pulsatile-release coating Modified release may be achieved by pH-dependent or pH-independent polymer coating.

In some embodiments the encapsulated or coated composition (e.g. a liquid or semisolid SEDDS, SMEDDS or SNEDDS comprising a GLP-1 peptide and a FA-aa) comprises less than 10% (w/w) water.

Coatings, such as enteric coatings, or modified release coatings may be prepared according to methods well known in the art.

### GLP-1 Peptides

In some embodiments the composition of the invention comprises a GLP-1 peptide, such as a GLP-1 analogue or a derivative thereof. In some embodiments the composition comprises at least one GLP-1 peptide. In some embodiments the GLP-1 peptide comprises a lipophilic side chain, such as a GLP-1 peptide comprising an alkyl moiety with at least 14 carbon atoms. In some embodiments the GLP-1 peptide is an acylated peptide. In some embodiments the GLP-1 peptide is acylated with a fatty acid or a fatty diacid. In some embodiments the GLP-1 peptide comprises substituent comprising a fatty acid or a fatty diacid, such as formula (X)

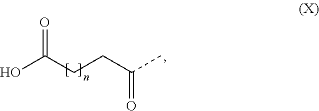

wherein n is at least 13. In some embodiments the GLP-1 peptide comprises one or more 8-amino-3,6-dioxaoctanoic acid (OEG).

The term “GLP-1 peptide” as used herein refers to a compound, which fully or partially activates the human GLP-1 receptor. In some embodiments the GLP-1 peptide binds to a GLP-1 receptor, e.g., with an affinity constant (K_{D}) or activate the receptor with a potency (EC₅₀) of below 1 μM, e.g. below 100 nM as measured by methods known in the art (see e.g. WO98/08871) and exhibits insulinotropic activity, where insulinotropic activity may be measured in vivo or in vitro assays known to those of ordinary skill in the art. For example, the GLP-1 peptide may be administered to an animal with increased blood glucose (e.g. obtained using an Intravenous Glucose Tolerance Test (IVGTT), a person skilled in the art will be able to determine a suitable glucose dosage and a suitable blood sampling regime, e.g. depending on the species of the animal, for the IVGTT) and the plasma insulin concentration measured over time. The biological activity of a GLP-1 peptide may be measured in an assay as known by a person skilled in the art, e.g. as described in WO98/08871.

In some embodiments the GLP-1 peptide is a GLP-1 analogue, optionally comprising one substituent. The term “analogue” as used herein referring to a GLP-1 peptide (hereafter “peptide”) means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified. Such addition or deletion of amino acid residues may take place at the N-terminal of the peptide and/or at the C-terminal of the peptide. In some embodiments a simple nomenclature is used to describe the GLP-1 peptide, e.g., [Aib8] GLP-1(7-37) designates an analogue of GLP-1(7-37) wherein the naturally occurring Ala in position 8 has been substituted with Aib. In some embodiments the GLP-1 peptide comprises a maximum of twelve, such as a maximum of 10, 8 or 6, amino acids which have been altered, e.g., by substitution, deletion, insertion and/or modification, compared to e.g. GLP-1(7-37). In some embodiments the analogue comprises up to 10 substitutions, deletions, additions and/or insertions, such as up to 9 substitutions, deletions, additions and/or insertions, up to 8 substitutions, deletions, additions and/or insertions, up to 7 substitutions, deletions, additions and/or insertions, up to 6 substitutions, deletions, additions and/or insertions, up to 5 substitutions, deletions, additions and/or insertions, up to 4 substitutions, deletions, additions and/or insertions or up to 3 substitutions, deletions, additions and/or insertions, compared to e.g. GLP-1(7-37). Unless otherwise stated the GLP-1 comprises only L-amino acids.

In some embodiments the term “GLP-1 analogue” or “analogue of GLP-1” as used herein refers to a peptide, or a compound, which is a variant of the human Glucagon-Like Peptide-1 (GLP-1(7-37)). GLP-1(7-37) has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or insertions.

In some embodiments the GLP-1 peptide exhibits at least 60%, 65%, 70%, 80% or 90% sequence identity to GLP-1(7-37) over the entire length of GLP-1(7-37). As an example of a method for determination of sequence identity between two analogues the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

In some embodiments the C-terminal of the GLP-1 peptide is an amide.

In some embodiments the GLP-1 peptide is GLP-1(7-37) or GLP-1(7-36)amide. In some embodiments the GLP-1 peptide is exendin-4, the sequence of which is HGEGTFITSDLSKQMEEEAVRLFIEWLKNGGPSSGAPPPS (SEQ ID No: 2).

In some embodiments the GLP-1 peptide comprises one substituent which is covalently attached to the peptide. In some embodiments the substituent comprises a fatty acid or a fatty diacid. In some embodiments the substituent comprises a C16, C18 or C20 fatty acid. In some embodiments the substituent comprises a C16, C18 or C20 fatty diacid. In some embodiments the substituent comprises formula (X)

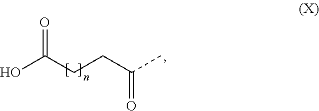

wherein n is at least 13, such as n is 13, 14, 15, 16, 17, 18 or 19. In some embodiments the substituent comprises formula (X), wherein n is in the range of 13 to 19, such as in the range of 13 to 17. In some embodiments the substituent comprises formula (X), wherein n is 13, 15 or 17. In some embodiments the substituent comprises formula (X), wherein n is 13. In some embodiments the substituent comprises formula (X), wherein n is 15. In some embodiments the substituent comprises formula (X), wherein n is 17. In some embodiments the substituent comprises one or more 8-amino-3,6-dioxaoctanoic acid (OEG), such as two OEG.

In some embodiments the substituent is [2-(2-{2-[2-(2-{2-[(S)-4-carboxy-4-(17-carboxyheptadecanoylamino)butyrylamino]ethoxy}ethoxy)acetylamino]ethoxy}ethoxy)acetyl].

In some embodiments the substituent is [2-(2-{2-[2-(2-{2-[(S)-4-carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl].

In some embodiments the GLP-1 peptide is semaglutide, also known as N-epsilon26-[2-(2-{2-[2-(2-{2-[(S)-4-carboxy-4-(17-carboxyheptadecanoylamino) butyrylamino]ethoxy}ethoxy)acetylamino]ethoxy}ethoxy)acetyl][Aib8,Arg34]GLP-1(7-37), which may be prepared as described in WO2006/097537, Example 4.

In some embodiments the composition comprises the GLP-1 peptide or a pharmaceutically acceptable salt, amide, or ester thereof. In some embodiments the composition comprises the GLP-1 peptide one or more pharmaceutically acceptable counter ions.

In some embodiments the amount of GLP-1 peptide is no more than 20% (w/w), such as no more than 15% (w/w) or no more than 10% (w/w), or such as 1-5% (w/w).

In some embodiments the dosage of GLP-1 is in the range of 0.01 mg to 100 mg. In some embodiments the composition comprises an amount of a GLP-1 peptide in the range of at least 1 mg, such as at least 5 mg or at least 10 mg. In some embodiments the composition comprises 10 mg GLP-1 peptide.

In some embodiments the composition comprises an amount of a GLP-1 peptide in the range of 0.05 to 25 μmol, such as in the range of 0.5 to 20 μmol.

In some embodiments the GLP-1 peptide is selected from one or more of the GLP-1 peptides mentioned in WO93/19175, WO96/29342, WO98/08871, WO99/43707, WO99/43706, WO99/43341, WO99/43708, WO2005/027978, WO2005/058954, WO2005/058958, WO2006/005667, WO2006/037810, WO2006/037811, WO2006/097537, WO2006/097538, WO2008/023050, WO2009/030738, WO2009/030771 and WO2009/030774.

In some embodiments the GLP-1 peptide is selected from the group consisting of N-epsilon37{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon26{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7, Arg34] GLP-1-(7-37); N-epsilon37{2-[2-(2-{2-[2-((S)-3-carboxy-3-{[1-(19-carboxy-nonadecanoyl) piperidine-4-carbonyl]amino}propionylamino)ethoxy]ethoxy}acetylamino)ethoxy]ethoxy}acetyl[Aib8,Glu22,Arg26,Arg34,Lys37]GLP-1-(7-37)amide; N-epsilon37-[2-(2-[2-(2-[2-(2-((R)-3-[1-(17-carboxyheptadecanoyl)piperidin-4-ylcarbonylamino]3-carboxypropionylamino)ethoxy)ethoxy]acetylamino)ethoxy]ethoxy)acetyl][,DesaminoHis7, Glu22 Arg26, Arg 34, Phe(m-CF3)28]GLP-1-(7-37)amide; N-epsilon26-[(S)-4-carboxy-4-({trans-4-[(19-carboxynonadecanoylamino)methyl]cyclohexanecarbonyl}amino)butyryl][Aib8,Arg34]GLP-1-(7-37); N-epsilon26-{4-[(S)-4-carboxy-4-({trans-4-[(19-carboxynonadecanoylamino) methyl]cyclohexanecarbonyl}amino)butyrylamino]butyryl}[Aib8,Arg34]GLP-1-(7-37); N-epsilon26-[2-(2-{2-[(S)-4-carboxy-4-({trans-4-[(19-carboxy-nonadecanoylamino) methyl]cyclohexanecarbonyl}amino)butyrylamino]ethoxy}ethoxy)acetyl][Aib8,Arg34]GLP-1-(7-37); N-epsilon26-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy) acetyl][Aib8,Arg34]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][Aib8,Glu22,Arg26, Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22, Arg26,Arg34,Lys37]GLP-1-(7-37)amide; N-epsilon37-[2-(2-{2-[2-(2-{2-[(S)-4-carboxy-4-({4-[(trans-19-carboxy-nonadecanoylamino)methyl]cyclohexanecarbonyl}amino) butyrylamino]ethoxy}ethoxy)acetylamino]ethoxy}ethoxy)acetyl][DesaminoHis7,Arg26,Arg34,L ys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22,Arg26,A rg34, Lys37]GLP-1-(7-37); N-epsilon26[2-(2-{2-[2-(2-{2-[(S)-4-carboxy-4-({4-[(19-carboxy- nonadecanoylamino)methyl]cyclohexanecarbonyl}amino)butyrylamino]ethoxy}ethoxy) acetylamino]ethoxy}ethoxy)acetyl[Aib8, Lys 26]GLP-1 (7-37)amide; N-epsilon26 [2-(2-[2-(2-[2-(2-((S)-2-[trans-4-((9-carboxynonadecanoylamino]methyl) cyclohexylcarbonylamino]-4-carboxybutanoylamino)ethoxy)ethoxy]acetylamino) ethoxy]ethoxy)acetyl][Aib8, Lys26] GLP-1 (7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl] [DesaminoHis7,Arg26,Arg34,Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][DesaminoH is7,Glu22, Arg26,Glu30,Arg34,Lys37]GLP-1-(7-37); N-epsilon26-[2-(2-{2-[(S)-4-carboxy-4-((S)-4-carboxy-4-{4-[4-(16-(1H-tetrazol-5-yl)-hexadecanoylsulfamoyl)butyrylamino]-butyrylamino}butyrylamino) butyrylamino]ethoxy}ethoxy)acetyl][Aib8,Arg34]GLP-1-(7-37); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino) butyrylamino]ethoxy}ethoxy)acetyl][Aib8,Arg34]GLP-1-(7-37); N-epsilon26-[2-(2-{2-[(S)-4-carboxy-4-((S)-4-carboxy-4-{6-[4-(16-(1H-tetrazol-5-yl)hexadecanoyl-sulfamoyl)butyrylamino]hexanoylamino}butyrylamino)butyrylamino]ethoxy}ethoxy) acetyl][Aib8,Arg34]GLP-1-(7-37); N-epsilon26-[2-(2-{2-[(S)-4-carboxy-4-((S)-4-carboxy-4-{4-[4-(16-(1H-tetrazol-5-yl)hexadecanoylsulfamoyl)butyrylamino]butyrylamino}butyrylamino)butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]-dodecanoylamino}butyrylamino) butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl] [Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-36)amide; N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyryl-amino)butyrylamino]ethoxy}ethoxy)acetyl][Aib8,Lys33,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino)butyrylamino]ethoxy}ethoxy)acetyl][Aib8,Arg34]GLP-1-(7-36)amide; N-epsilon26-[2-(2-{2-[2-(2-{2-[2-(2-{2-[2-(2-{2-[2-(2-{2-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy) acetylamino]ethoxy}ethoxy)acetylamino]ethoxy}ethoxy)acetylamino]ethoxy}ethoxy)acetylami no]ethoxy}ethoxy)acetylamino]ethoxy}ethoxy)acetyl][Aib8,Lys26,Arg34]GLP-1-(7-36)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][DesaminoHis7,Glu22,Arg26,Arg34,Lys37]GLP-1-(7-37)amide; N-epsilon37{2-[2-(2-{2-[2-((R)-3-carboxy-3-{[1-(19-carboxy-nonadecanoyl) piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon37{2-[2-(2-{2-[2-((S)-3-carboxy-3-{[1-(19-carboxynonadecanoyl) piperidine-4-carbonyl]amino}propionylamino) ethoxy]ethoxy}acetylamino)ethoxy]ethoxy}acetyl [Aib8,Glu22, Arg26,Arg34, Lys37]GLP-1-(7-37)amide; N-epsilon37-[2-(2-[2-(2-[2-(2-((R)-3-[1-(17-carboxyhepta-decanoyl)piperidin-4-ylcarbonylamino]3-carboxy-propionylamino) ethoxy)ethoxy]acetylamino) ethoxy]ethoxy)acetyl] [DesaminoHis7, Glu22,Arg26, Arg34,Phe(m-CF3)28] GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino] ethoxy}ethoxy)acetyl] [Aib8,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy) acetylamino]ethoxy}ethoxy)acetyl] [DesaminoHis7,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy) acetylamino]ethoxy}ethoxy)acetyl] [DesaminoHis7,Glu22,Arg26,Arg34, Lys37]GLP-1-(7-37); N-epsilon37-[2-(2-{2-[2-(2-{2-[(S)-4-carboxy-4-({trans-4-[(19-carboxy-nonadecanoylamino) methyl]cyclohexanecarbonyl}amino)butyrylamino]ethoxy}ethoxy) acetylamino]ethoxy}ethoxy)acetyl] [DesaminoHis7,Glu22,Arg26,Glu30,Arg34, Lys37]GLP-1-(7-37); N-epsilon37-[2-(2-{2-[(S)-4-carboxy-4-((S)-4-carboxy-4-{12-[4-(16-(1H-tetrazol-5-yl)hexadecanoyl-sulfamoyl) butyrylamino]dodecanoylamino}butyrylamino) butyrylamino]ethoxy}ethoxy)acetyl] [Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy)acetyl] [DesaminoHis7,Glu22,Arg26,Arg34,Lys37]GLP-1-(7-37)amide; N-epsilon37-(3-((2-(2-(2-(2-(2-Hexadecyloxyethoxy)ethoxy)ethoxy) ethoxy) ethoxy)) propionyl)[DesaminoHis7,Glu22,Arg26,Arg34,Lys37]GLP-1(7-37)-amide; N-epsilon37-{2-(2-(2-(2-[2-(2-(4-(hexadecanoylamino)-4-carboxybutyryl-amino)ethoxy) ethoxy]acetyl)ethoxy)ethoxy)acetyl)}]-[desaminoHis7,Glu22,Arg26, Glu30,Arg34,Lys37] GLP-1-(7-37)amide; N-epsilon37-{2-(2-(2-(2-[2-(2-(4-(hexadecanoylamino)-4-carboxy-butyryl-amino) ethoxy)ethoxy]acetyl)ethoxy)ethoxy)acetyl)}-[desaminoHis7,Glu22, Arg26, Arg34,Lys37]GLP-1-(7-37)amide; N-epsilon37-(2-(2-(2-(2-(2-(2-(2-(2-(2-(octadecanoyl-amino)ethoxy)ethoxy) acetylamino)ethoxy) ethoxy)acetylamino) ethoxy)ethoxy) acetyl)[desaminoHis7,Glu22,Arg26,Arg34,Lys37] GLP-1 (7-37)amide; N-epsilon37-[4-(16-(1H-Tetrazol-5-yl)hexadecanoylsulfamoyl) butyryl] [DesaminoHis7,Glu22,Arg26, Arg34, Lys37]GLP-1-(7-37)amide; N-epsilon37-[2-(2-{2-[2-(2-{2-[(S)-4-carboxy-4-(19-carboxynonadecanoylamino) butyrylamino]ethoxy}ethoxy) acetylamino]ethoxy}ethoxy)acetyl] [DesaminoHis7,Glu22,Arg26, Arg34,Lys37]GLP-1-(7-37); N-epsilon37-(2-{2-[2-((S)-4-carboxy-4-{(S)-4-carboxy-4-[(S)-4-carboxy-4-(19-carboxy-nonadecanoylamino)butyrylamino]butyrylamino}butyrylamino)ethoxy]ethoxy} acetyl)[DesaminoHis7,Glu22,Arg26,Arg34,Lys37]GLP-1-(7-37); N-epsilon37-{2-[2-(2-{(S)-4-[(S)-4-(12-{4-[16-(2-tert-Butyl-2H-tetrazol-5-yl)-hexadecanoylsulfamoyl]butyrylamino}dodecanoylamino)-4-carboxybutyrylamino]-4-carboxybutyrylamino} ethoxy)ethoxy]acetyl}[DesaminoHis7,Glu22,Arg26,Arg34,Lys37] GLP-1 (7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [Aib8,Glu22, Arg26,Arg34,Lys37]GLP-1-(7-37); N-alpha37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [Aib8,Glu22,Arg26,Arg34,epsilon-Lys37]GLP-1-(7-37)peptide; N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [desaminoHis7, Glu22,Arg26,Arg34,Lys37] GLP-1-(7-37); N-epsilon36-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [desaminoHis7, Glu22,Arg26,Glu30,Arg34,Lys36] GLP-1-(7-37)-Glu-Lys peptide; N-epsilon37-[2-(2-{2-[2-(2-{2-[(S)-4-carboxy-4-({trans-4-[(19-carboxynonadecanoylamino)methyl]cyclohexanecarbonyl}amino)butyryl-amino]ethoxy}ethoxy)acetylamino]ethoxy}ethoxy)acetyl][Aib8,Glu22,Arg26,Arg34,Lys37]GLP-1-(7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylHAib8,Glu22, Arg26,Arg34,Aib35,Lys37]GLP-1-(7-37); N-epsilon37-[(S)-4-carboxy-4-(2-{2-[2-(2-{2-[2-(17-carboxyheptadecanoylamino)ethoxy]ethoxy}acetylamino) ethoxy]ethoxy}acetylamino) butyryl] [Aib8,Glu22,Arg26,34,Lys37] GLP-1 (7-37); N-epsilon37-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [ImPr7,Glu22, Arg26,34,Lys37], GLP-1-(7-37); N-epsilon26-{2-[2-(2-{2-[2-(2-{(S)-4-carboxy-4-[10-(4-carboxyphenoxyl) decanoylamino]butyrylamino}ethoxy)ethoxy]acetylamino}ethoxy) ethoxy]acetyl}, N-epsilon37-{2-[2-(2-{2-[2-(2-{(S)-4-carboxy-4-[10-(4-carboxy-phenoxy) decanoylamino]butyrylamino}ethoxy)ethoxy]acetylamino}ethoxy) ethoxy]acetyl}-[Aib8,Arg34,Lys37]GLP-1(7-37)-OH; N-epsilon26 (17-carboxyhepta-decanoyl)-[Aib8,Arg34]GLP-1-(7-37)-peptide; N-epsilon26-(19-carboxynonadecanoyl)-[Aib8,Arg34]GLP-1-(7-37); N-epsilon26-(4-{[N-(2-carboxyethyl)-N-(15-carboxypenta-decanoyl)amino]methyl}benzoyl[Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl][Aib8,Arg34]GLP-1-(7-37); N-epsilon26-[2-(2-[2-(2-[2-(2-[4-(19-carboxynonadecanoylamino)-4(S)-carboxybutyrylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][3-(4-Imidazolyl)Propionyl7,Arg34]GLP-1-(7-37); N-epsilon26-[2-(2-[2-(2-[2-(2-[4-(17-carboxyheptadecanoylamino)-(carboxymethyl-amino)acetylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-3(S)-Sulfopropionylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Gly8,Arg34] GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34]GLP-1-(7-37)-amide; N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [Aib8,Arg34,Pro37]GLP-1-(7-37)amide; Aib8,Lys26(N-epsilon26-{2-(2-(2-(2-[2-(2-(4-(pentadecanoylamino)-4-carboxybutyrylamino)ethoxy)ethoxy]acetyl)ethoxy) ethoxy)acetyl)}), Arg34)GLP-1H(7-37)-OH; N-epsilon26-[2-(2-[2-(2-[2-(2-[4-{[N-(2-carboxyethyl)-N-(17-carboxyheptadecanoyl)amino]methyl}benzoyl)amino]ethoxy) ethoxy]acetylamino)ethoxy]ethoxy)acetyl][Aib8,Arg34]GLP-1(7-37); N-alpha7-formyl, N-epsilon26-[2-(2-[2-(2-[2-(2-[4-(17-carboxyheptadecanoyl-amino)-4(S)-carboxybutyrylamino]ethoxy)ethoxy]acetylamino)ethoxy]ethoxy)acetyl] [Arg34]GLP-1-(7-37); N-epsilon2626-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8, Glu22, Arg34] GLP-1-(7-37); N-epsilon26{3-[2-(2-{2-[2-(2-{2-[2-(2-[4-(15-(N—((S)-1,3-dicarboxypropyl) carbamoyl)pentadecanoylamino)-(S)-4-carboxybutyrylamino]ethoxy)ethoxy]ethoxy}ethoxy)ethoxy]ethoxy}ethoxy)ethoxy]propionyl} [Aib8,Arg34]GLP-1-(7-37); N-epsilon26-[2-(2-[2-(2-[2-(2-[4-{[N-(2-carboxyethyl)-N-(17-carboxy-heptadecanoyl)amino]methyl}benzoyl)amino](4(S)-carboxybutyryl-amino)ethoxy) ethoxy]acetylamino)ethoxy]ethoxy)acetyl][Aib8,Arg34] GLP-1(7-37); N-epsilon26-{(S)-4-carboxy-4-((S)-4-carboxy-4-((S)-4-carboxy-4-((S)-4-carboxy-4-(19-carboxy-nonadecanoylamino)butyrylamino)butyrylamino)butyrylamino) butyrylamino} [Aib8,Arg34]GLP-1-(7-37); N-epsilon26-4-(17-carboxyheptadecanoyl-amino)-4(S)-carboxybutyryl-[Aib8,Arg34]GLP-1-(7-37); N-epsilon26-{3-[2-(2-{2-[2-(2-{2-[2-(2-[4-(17-carboxyheptadecanoylamino)-4(S)-carboxybutyrylamino]ethoxy)ethoxy]ethoxy}ethoxy)ethoxy]ethoxy}ethoxy)ethoxy]propionyl}[Aib8,Arg34]GLP-1-(7-37); N-epsilon26-{2-(2-(2-(2-[2-(2-(4-(17-carboxyheptadecanoylamino)-4-carboxybutyrylamino) ethoxy)ethoxy]acetyl)ethoxy)ethoxy)acetyl)}-[Aib8,22,27,30,35,Arg34,Pro37, Lys26] GLP-1 (7-37)amide; N-epsilon26-[2-(2-[2-[4-(21-carboxyuneicosanoylamino)-4(S)-carboxybutyrylamino]ethoxy]ethoxy)acetyl][Aib8,Arg34]GLP-1-(7-37); and N-epsilon26-[2-(2-[2-(2-[2-(2-[4-(21-carboxyuneicosanoylamino)-4(S)-carboxybutyrylamino]ethoxy)ethoxy]acetylamino)ethoxy]ethoxy)acetyl][Aib8,Arg34]GLP-1-(7-37).

In some embodiments the GLP-1 peptide is N-epsilon26-[2-(2-{2-[2-(2-{2-[(S)-4-carboxy-4-(17-carboxyheptadecanoylamino)butyrylamino]ethoxy}ethoxy)acetylamino]ethoxy}ethoxy)acetyl][Aib8,Arg34]GLP-1(7-37), also known as semaglutide.

In some embodiments GLP-1 peptides may be produced by appropriate derivatisation of an appropriate peptide backbone which has been produced by recombinant DNA technology or by peptide synthesis (e.g., Merrifield-type solid phase synthesis) as known in the art of peptide synthesis and peptide chemistry.

In some embodiments the production of peptides like GLP-1(7-37) and GLP-1 analogues is well known in the art. The GLP-1 moiety of the GLP-1 peptide of the invention (or fragments thereof) may for instance be produced by classical peptide synthesis, e.g., solid phase peptide synthesis using t-Boc or Fmoc chemistry or other well established techniques, see, e.g., Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000.

In some embodiments GLP-1 peptides may be produced by recombinant methods, viz. by culturing a host cell containing a DNA sequence encoding the GLP-1 peptide and capable of expressing the peptide in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

In some embodiments GLP-1 peptides of the invention which include non-natural amino acids and/or a covalently attached N-terminal mono- or dipeptide mimetic may e.g. be produced as described in the experimental part, or see e.g., Hodgson et al: “The synthesis of peptides and proteins containing non-natural amino acids”, Chemical Society Reviews, vol. 33, no. 7 (2004), p. 422-430; and WO 2009/083549 A1 entitled “Semi-recombinant preparation of GLP-1 analogues”.

In some embodiments the GLP-1 peptide has a solubility of at least 50 mg/mL, such as at least 60 mg/mL, at least 70 mg/mL, or at least 80 mg/mL, such as at least 90 mg/mL, at least 100 mg/mL, or at least 110 mg/mL, such as at least 120 mg/mL, at least 130 mg/mL, or at least 140 mg/mL in water, such as at least 150/mL, at least 160 mg/mL, or at least 170 mg/mL, such as at least 180 mg/mL, at least 190 mg/mL, or at least 200 mg/mL, such as at least 210 mg/mL, at least 220 mg/mL, or at least 230 mg/mL, or such as at least 240 mg/mL in aqueous sodium phosphate buffer at pH 8.5 and room temperature.

In some embodiments the GLP-1 peptide has a protracted pharmacokinetic profile, i.e. the GLP-1 peptide protracted. In some embodiments protraction may be determined as half-life (T_{1/2}) in vivo in minipigs after i.v. administration. In some embodiments the half-life is at least 24 hours, such as at least 48 hours, at least 60 hours, at least 72 hours, or such as at least 84 hours, at least 96 hours, or at least 108 hours. A suitable assay for determining half-life in vivo in minipigs after i.v. administration is the following, wherein the purpose is to determine the protraction in vivo of GLP-1 agonists after i.v. administration to minipigs, i.e. the prolongation of their time of action; this is done in a pharmacokinetic (PK) study, where the terminal half-life of the GLP-1 agonist in question is determined. By terminal half-life is generally meant the period of time it takes to halve a certain plasma concentration, measured after the initial distribution phase. Male Gottingen minipigs are obtained from Ellegaard Gottingen Minipigs (Dalmose, Denmark) approximately 7-14 months of age and weighing from approximately 16-35 kg are used in the studies. The minipigs are housed individually and fed restrictedly once or twice daily with SDS minipig diet (Special Diets Services, Essex, UK). After at least 2 weeks of acclimatisation two permanent central venous catheters are implanted in vena cava caudalis or cranialis in each animal. The animals are allowed 1 week recovery after the surgery, and are then used for repeated pharmacokinetic studies with a suitable wash-out period between dosings. The animals are fasted for approximately 18 h before dosing and for at least 4 h after dosing, but have ad libitum access to water during the whole period. The GLP-1 agonist is dissolved in 50 mM sodium phosphate, 145 mM sodium chloride, 0.05% tween 80, pH 7.4 to a concentration of usually from 20-60 nmol/ml. Intravenous injections (the volume corresponding to usually 1-2 nmol/kg, for example 0.033 ml/kg) of the compounds are given through one catheter, and blood is sampled at predefined time points for up till 13 days post dosing (preferably through the other catheter). Blood samples (for example 0.8 ml) are collected in EDTA buffer (8 mM) and then centrifuged at 4° C. and 1942 G for 10 minutes. Plasma is pippetted into Micronic tubes on dry ice, and kept at −20° C. until analyzed for plasma concentration of the respective GLP-1 compound using ELISA or a similar antibody based assay or LC-MS. Individual plasma concentration-time profiles are analyzed by a non-compartmental model in WinNonlin v. 5.0 (Pharsight Inc., Mountain View, Calif., USA), and the resulting terminal half-lives (harmonic mean) determined.

### Pharmaceutical Indications

The present invention also relates to a composition of the invention for use as a medicament. In some embodiments the composition is administered orally. In some embodiments the composition is administered to a subject, such as a human.

In particular embodiments the composition of the invention may be used for the following medical treatments, all preferably relating one way or the other to diabetes:

(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;

(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;

(iii) improving β-cell function, such as decreasing β-cell apoptosis, increasing β-cell function and/or β-cell mass, and/or for restoring glucose sensitivity to β-cells;

(iv) prevention and/or treatment of cognitive disorders;

(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying;

(vi) prevention and/or treatment of diabetic complications, such as neuropathy, including peripheral neuropathy; nephropathy; or retinopathy;

(vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein a (apo(a)) in vitro and/or in vivo;

(iix) prevention and/or treatment of cardiovascular diseases, such as syndrome X; atherosclerosis; myocardial infarction; coronary heart disease; stroke, cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atheroschlerosis; mild chronic heart failure; angina pectoris; cardiac bypass reocclusion; intermittent claudication (atheroschlerosis oblitterens); diastolic dysfunction; and/or systolic dysfunction;

(ix) prevention and/or treatment of gastrointestinal diseases, such as inflammatory bowel syndrome; small bowel syndrome, or Crohn's disease; dyspepsia; and/or gastric ulcers;

(x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or

(xi) prevention and/or treatment of polycystic ovary syndrome (PCOS).

In a particular embodiment, the indication is selected from the group consisting of (i)-(iii) and (v)-(iix), such as indications (i), (ii), and/or (iii); or indication (v), indication (vi), indication (vii), and/or indication (iix).

In another particular embodiment, the indication is (i). In a further particular embodiment the indication is (v). In a still further particular embodiment the indication is (iix).

In some embodiments the invention relates to a composition of the invention for treatment of diabetes or obesity, wherein said composition is administered orally. In some embodiments the invention relates to a method for treatment of diabetes or obesity comprising oral administration of a composition of the invention to a patient in need thereof.

The following indications are particularly preferred: Type 2 diabetes and/or obesity.

In some embodiments the term “comprise” means “consist of”. In some embodiments “at least one” means one.

### Embodiments of the Invention

The following are non-limiting embodiments of the invention:
1. A pharmaceutical composition comprising i) a GLP-1 peptide and ii) at least one fatty acid amino acid (FA-aa) or a salt of said FA-aa.
2. The pharmaceutical composition according to any of the preceding embodiments, wherein the composition is an oral pharmaceutical composition.
3. The pharmaceutical composition according to any of the preceding embodiments, wherein said FA-aa comprises an amino acid acylated at a free amino group with a fatty acid, wherein said fatty acid comprises an alkyl group consisting of 5 to 19 carbon atoms.
4. The pharmaceutical composition according to any of the preceding embodiments, wherein said
   a. FA-aa has the general formula I:

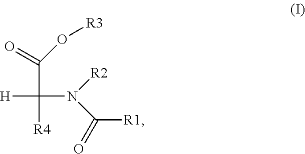

wherein
R1 is an alkyl group consisting of 5 to 19 carbon atoms;
   R2 is H (i.e. hydrogen), CH₃ (i.e. methyl group), or covalently attached to R4 via a (CH₂)₃ group;
      R3 is H or absent; and
      R4 is an amino acid side chain, or covalently attached to R2 via a (CH₂)₃ group.
5. The pharmaceutical composition according to any of the preceding embodiments, wherein R2 and R4 are covalently attached via a (CH₂)₃ group.
6. The pharmaceutical composition according to any of the preceding embodiments, wherein R1 is an alkyl group consisting of 7 to 17 carbon atoms, such as 9-15-carbon atoms or 11-13 carbon atoms.
7. The pharmaceutical composition according to any of the preceding embodiments, wherein said GLP-1 peptide is a GLP-1 analogue or a derivative thereof comprising less than 10 substitutions, deletions or insertions compared to human GLP-1(7-37).
8. The pharmaceutical composition according to any of the preceding embodiments, wherein said GLP-1 peptide is an acylated GLP-1 peptide, such as semaglutide.
9. The pharmaceutical composition according to any of the preceding embodiments, wherein said composition comprises less than 10% (w/w) water.
10. The pharmaceutical composition according to any of the preceding embodiments, wherein R4 is an amino acid side chain selected from the group consisting of a non-cationic amino acid side chain, a non-polar hydrophobic amino acid side chain, a polar non-charged amino acid side chain, or a polar acidic amino acid side chain.
11. The pharmaceutical composition according to any of the preceding embodiments, wherein said FA-aa comprises an amino acid residue selected from the group consisting of a non-cationic amino acid residue, a non-polar hydrophobic amino acid residue, a polar non-charged amino acid residue, or a polar acidic amino acid residue.
12. The pharmaceutical composition according to any of the preceding embodiments, wherein said FA-aa is in the form of its free acid or the salt thereof, such as the sodiumsalt.
13. The pharmaceutical composition according to any of the preceding embodiments, wherein said alkyl group consists of 9, 11, 13, 15, or 17 carbon atoms.
14. The pharmaceutical composition according to any of the preceding embodiments, wherein said alkyl group consists of 9, 11, or 13 carbon atoms.
15. The pharmaceutical composition according to any of the preceding embodiments, wherein said alkyl group consists of 13, 15, or 17 carbon atoms.
16. The pharmaceutical composition according to any of the preceding embodiments, wherein the amino acid residue of said FA-aa is the amino acid residue of an amino acid selected from the group consisting of Alanine (Ala), Valine (Val), Leucine (Leu), Isoleucine (Ile), Phenylalanine (Phe), Tryptophane (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp) and Glutamic acid (Glu).
17. The pharmaceutical composition according to any of the previous embodiments, wherein the amino acid residue is a sarcosine residue, a glutamic acid residue, or a leucine residue.
18. The pharmaceutical composition according to any of the previous embodiments, wherein R1 is H and R4 is methyl.
19. The pharmaceutical composition according to any of the previous embodiments, wherein R1 is —(CH₂)₂COOH and R4 is H.
20. The pharmaceutical composition according to any of the previous embodiments, wherein R1 is —(CH₂)₂(CH)(CH) and R4 is H.
21. The pharmaceutical composition according to any of the preceding embodiments, wherein the FA-aa is selected from the group consisting of sodium N-dodecanoyl alaninate, N-dodecanoyl-L-alanine, sodium N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutamic acid, N-dodecanoyl-L-glutamic acid, sodium N-dodecanoyl glutaminate, N-dodecanoyl-L-glutamine, sodium N-dodecanoyl glycinate, N-dodecanoyl-L-glycine, sodium dodecanoyl histidinate, N-dodecanoyl-L-histidine, sodium dodecanoyl isoleucinate, N-dodecanoyl-L-isoleucine, sodium dodecanoyl leucinate, N-dodecanoyl-L-leucine, sodium dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl serinate, N-dodecanoyl-L-serine, sodium N-dodecanoyl threoninate, N-dodecanoyl-L-threonine, sodium N-dodecanoyl tryptophanate, N-dodecanoyl-L-tryptophane, sodium N-dodecanoyl tyrosinate, N-dodecanoyl-L-tyrosine, sodium N-dodecanoyl valinate, N-dodecanoyl-L-valine, sodium N-dodecanoyl sarcosinate, N-dodecanoyl-L-sarcosine, sodium N-decanoyl alaninate, N-decanoyl-L-alanine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutamic acid, N-decanoyl-L-glutamic acid, sodium N-decanoyl glutaminate, N-decanoyl-L-glutamine, sodium N-decanoyl glycinate, N-decanoyl-L-glycine, sodium N-decanoyl histidinate, N-decanoyl-L-histidine, sodium N-decanoyl isoleucinate, N-decanoyl-L-isoleucine, sodium N-decanoyl leucinate, N-decanoyl-L-leucine, sodium N-decanoyl methioninate, N-decanoyl-L-methionine, sodium N-decanoyl phenylalaninate, N-decanoyl-L-phenylalanine, sodium N-decanoyl prolinate, N-decanoyl-L-proline, sodium N-decanoyl serinate, N-decanoyl-L-serine, sodium N-decanoyl threoninate, N-decanoyl-L-threonine, sodium N-decanoyl tryptophanate, N-decanoyl-L-tryptophane, sodium N-decanoyl tyrosinate, N-decanoyl-L-tyrosine, sodium N-decanoyl valinate, N-decanoyl-L-valine, sodium N-decanoyl sarcosinate, and N-decanoyl-L-sarcosine, sodium N-dodecanoyl sarcosinate, sodium N-oleoyl sarcosinate, sodium N-decyl leucine, Amisoft HS-11 P (sodium Stearoyl Glutamate, Amisoft MS-11 (sodium Myristoyl Glutamate)), Amisoft LS-11 (sodium Dodecanoyl Glutamate), Amisoft CS-11 (sodium Cocoyl Glutamate), Amilite GCS-11 (sodium Cocoyl Glycinate), sodium N-dodecanoyl sarcosinate, sodium N-decyl leucine, sodium N-cocoyl glycine, sodium N-cocoyl glutamate sodium N-dodecanoyl alaninate, N-dodecanoyl-L-alanine, sodium N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutamic acid, N-dodecanoyl-L-glutamic acid, sodium N-dodecanoyl glutaminate, N-dodecanoyl-L-glutamine, sodium N-dodecanoyl glycinate, N-dodecanoyl-L-glycine, sodium N-dodecanoyl histidinate, N-dodecanoyl-L-histidine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl serinate, N-dodecanoyl-L-serine, sodium N-dodecanoyl threoninate, N-dodecanoyl-L-threonine, sodium N-dodecanoyl tryptophanate, N-dodecanoyl-L-tryptophane, sodium N-dodecanoyl tyrosinate, N-dodecanoyl-L-tyrosine, sodium N-dodecanoyl valinate, N-dodecanoyl-L-valine, sodium N-dodecanoyl sarcosinate, N-dodecanoyl-L-sarcosine, sodium N-decanoyl alaninate, N-decanoyl-L-alanine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutamic acid, N-decanoyl-L-glutamic acid, sodium N-decanoyl glutaminate, N-decanoyl-L-glutamine, sodium N-decanoyl glycinate, N-decanoyl-L-glycine, sodium N-decanoyl histidinate, N-decanoyl-L-histidine, sodium N-decanoyl isoleucinate, N-decanoyl-L-isoleucine, sodium N-decanoyl leucinate, N-decanoyl-L-leucine, sodium N-decanoyl methioninate, N-decanoyl-L-methionine, sodium N-decanoyl phenylalaninate, N-decanoyl-L-phenylalanine, sodium N-decanoyl prolinate, N-decanoyl-L-proline, sodium N-decanoyl serinate, N-decanoyl-L-serine, sodium N-decanoyl threoninate, N-decanoyl-L-threonine, sodium N-decanoyl tryptophanate, N-decanoyl-L-tryptophane, sodium N-decanoyl tyrosinate, N-decanoyl-L-tyrosine, sodium N-decanoyl valinate, N-decanoyl-L-valine, sodium N-decanoyl sarcosinate and N-decanoyl-L-sarcosine, sodium N-dodecanoyl sarcosinate, sodium N-oleoyl sarcosinate, sodium N-decyl leucine, Amisoft HS-11 P (sodium Stearoyl Glutamate, Amisoft MS-11 (sodium Myristoyl Glutamate)), Amisoft LS-11 (sodium Dodecanoyl Glutamate), Amisoft CS-11 (sodium Cocoyl Glutamate), Amilite GCS-11 (sodium Cocoyl Glycinate), sodium N-dodecanoyl sarcosinate, sodium N-decyl leucine, sodium N-cocoyl glycine, and sodium N-cocoyl glutamate.
22. The pharmaceutical composition according to any of the preceding embodiments, wherein the FA-aa is N-decyl leucine, such as sodium N-decyl L-leucinate.
23. The pharmaceutical composition according to any of the preceding embodiments, wherein the FA-aa is N-dodecanoyl sarcosine, such as sodium N-dodecanoyl sarcosinate.
24. The pharmaceutical composition according to any of the preceding embodiments, wherein the FA-aa is N-myristoyl glutamine, such as sodium N-myristoyl L-glutamate or disodium N-myristoyl L-glutamate.
25. The pharmaceutical composition according to any of the preceding embodiments, wherein said composition comprises propylene glycol.
26. The pharmaceutical composition according to any of the preceding embodiments, wherein said composition comprises SEDDS, SMEDDS or SNEDDS.
27. The pharmaceutical composition according to any of the preceding embodiments, wherein said composition comprises one or more additional pharmaceutically acceptable excipients.
28. The pharmaceutical composition according to any of the preceding embodiments, wherein said composition is in the form of a solid, a liquid, or a semisolid.
29. The pharmaceutical composition according to any of the preceding embodiments, wherein said composition is in the form of a tablet or a multiparticulate.
30. The pharmaceutical composition according to any of the preceding embodiments, wherein said composition is in the form of a capsule.
31. The pharmaceutical composition according to any of the preceding embodiments, wherein said composition further comprises an enteric or modified release coating.
32. The pharmaceutical composition as defined in any of the preceding embodiments for use as a medicament.
33. The pharmaceutical composition as defined in any of the preceding embodiments for treatment and/or prevention of diabetes.
34. Use of the pharmaceutical composition as defined in any of the preceding embodiments for increasing the oral bioavailability of a GLP-1 peptide.
35. A method for increasing bioavailability of a GLP-1 peptide comprising oral administration of the pharmaceutical composition as defined in any of the preceding embodiments to a subject.
36. A method for increasing bioavailability of a GLP-1 peptide comprising the steps of including a FA-aa in a pharmaceutical composition of a GLP-1 peptide administered to a subject.
37. A method for increasing the plasma concentration of a GLP-1 peptide comprising the step of exposing the gastrointestinal tract of a subject to a pharmaceutical composition comprising a GLP-1 peptide and a FA-aa resulting in an increased plasma concentration of said GLP-1 peptide in said subject.
38. The method of embodiment 37, wherein said exposure is achieved by oral administration of said pharmaceutical composition.
39. A method for increasing the uptake of a GLP-1 peptide comprising the step of: exposing the gastrointestinal tract of a subject to a GLP-1 peptide and at least one FA-aa, whereby the plasma concentration of said GLP-1 peptide in said subject is increased compared to an exposure not including the at least one FA-aa.
40. A method for increasing uptake of a GLP-1 peptide across an/the epithelia cell layer of the gastro intestinal tract comprising the steps of, administering a pharmaceutical composition comprising a GLP-1 peptide and at least one FA-aa to a subject, whereby an increased uptake of said GLP-1 peptide is obtained compared to the uptake of said GLP-1 peptide obtained when said GLP-1 peptide composition does not including the at least one FA-aa.
41. The method of embodiments 35-40, where in the pharmaceutical composition is described by any one of embodiments 1-33.

### EXAMPLES

### Materials and Methods

Semaglutide may be prepared as described in WO2006/097537, Example 4.

### General Methods of Detection and Characterisation

### Assay (I): Pharmacokinetics Study in Rats

Animals, Dosing and Blood Sampling:

Male Sprague Dawley rats were used in the study. The rats were fasted on grid for approximately 18 h before the experiment and taken into general anaesthesia. The abdomen was opened in the midline and the intestine was arranged so that the jejunum was exposed. A catheter was inserted into the jejunum approximately 50 cm proximal to the cecum. The catheter was forwarded at least 1.5 cm into the jejunum, and secured before dosing by ligating around the gut and the catheter with suture to prevent leak or catheter displacement. A 1 ml syringe mounted with a 23 G needle was used for dosing of 100 μl of dosing solution into the intestine via the catheter at time=0 minutes. For dosing of tablets, the catheters were loaded with tablets prior and ejected into the intestine via a metal plunger at time t=0 minutes. Blood samples were taken at the following time points: 30, 60, 120 and 180 minutes after dosing.

Preparation of Plasma:

All blood samples were collected into test tubes containing EDTA for stabilisation and kept on ice until centrifugation. Plasma was separated from whole blood by centrifugation and the plasma was stored at −20° C. or lower until analysis.

Analysis of Plasma Samples:

The plasma was analysed for semaglutide using a Luminescence Oxygen Channeling Immunoassay (LOCI). The LOCI assay employs donor beads coated with streptavidin and acceptor beads conjugated with a monoclonal antibody binding to a mid-molecular region of semaglutide. The other monoclonal antibody, specific for an N-terminal epitope, was biotinylated. In the assay the three reactants were combined with 15 the semaglutide which form a two-sited immuno-complex. Illumination of the complex releases singlet oxygen atoms from the donor beads which channels into the acceptor beads and trigger chemiluminescence which was measured in the EnVision plate reader. The amount of light was proportional to the concentration of semaglutide and the lower limit of quantification (LLOQ) in plasma was 100 pM.

### Example 1

### Aqueous Compositions with Fatty Acid Acylated Amino Acid

The purpose of this experiment was to determine oral bioavailability of a GLP-1 peptide in an aqueous composition comprising a fatty acid acylated amino acid.

### Preparation of Compositions

Semaglutide (1000 nmol/ml) and a fatty acid acylated amino acid selected from the group consisting of sodium N-dodecanoyl sarcosinate, sodium N-decyl L-leucine, sodium N-cocoyl L-glycine, sodium N-cocoyl L-glutamate, sodium N-dodecanoyl L-glutamate, sodium N-myristoyl L-glutamate (55 mg/ml) were dissolved directly in water. Then the solution was adjusted to pH 8-8.5 and it was observed that the composition was clear.

### Rat Pharmacokinetic Experiment

Plasma exposure and area under the curve (AUC) of 55 mg/ml sodium N-dodecanoyl sarcosinate, sodium N-decyl L-leucine, sodium N-cocoyl L-glycine, sodium N-cocoyl L-glutamate, sodium N-dodecanoyl L-glutamate, sodium N-myristoyl L-glutamate was determined as described in Assay (I).

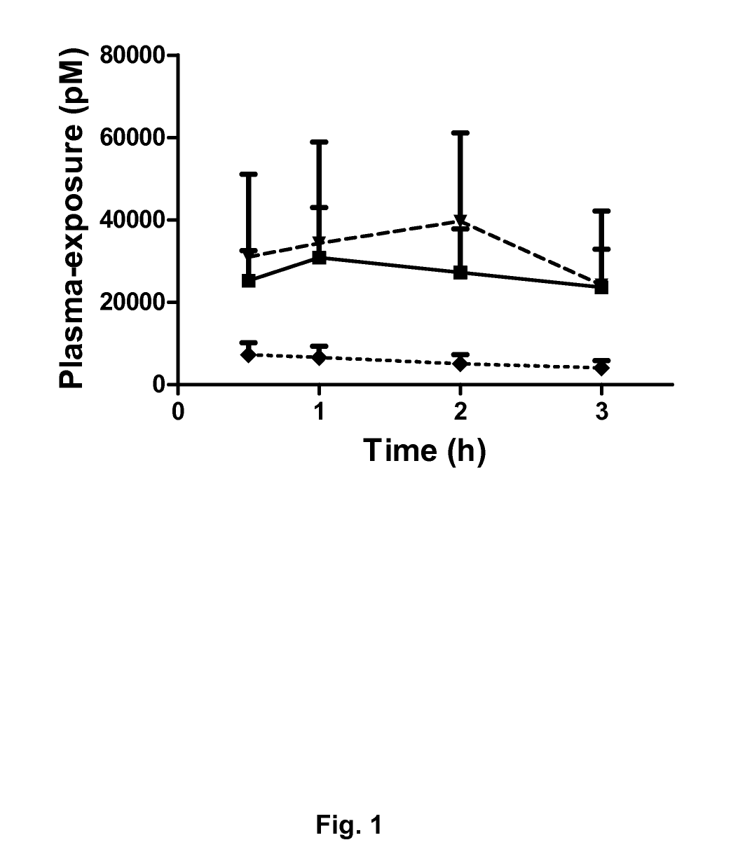

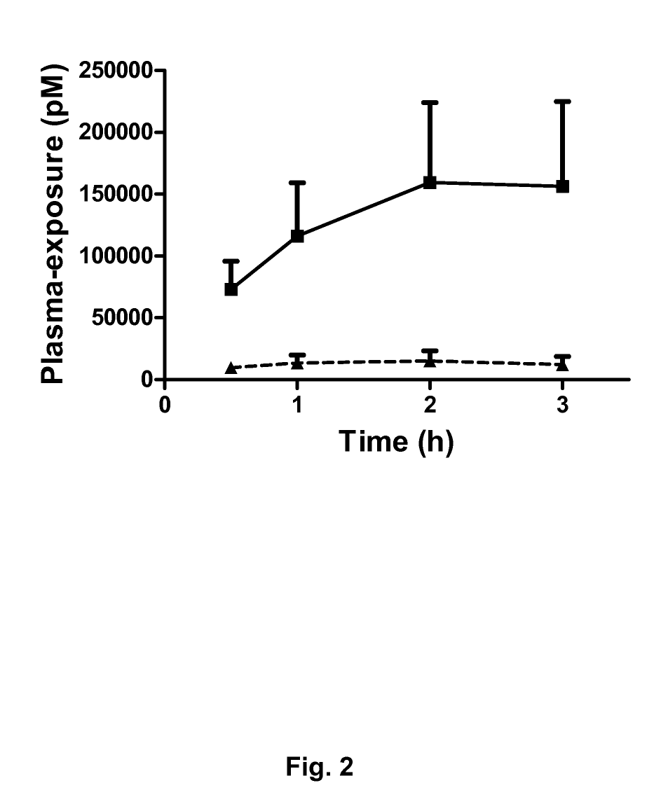

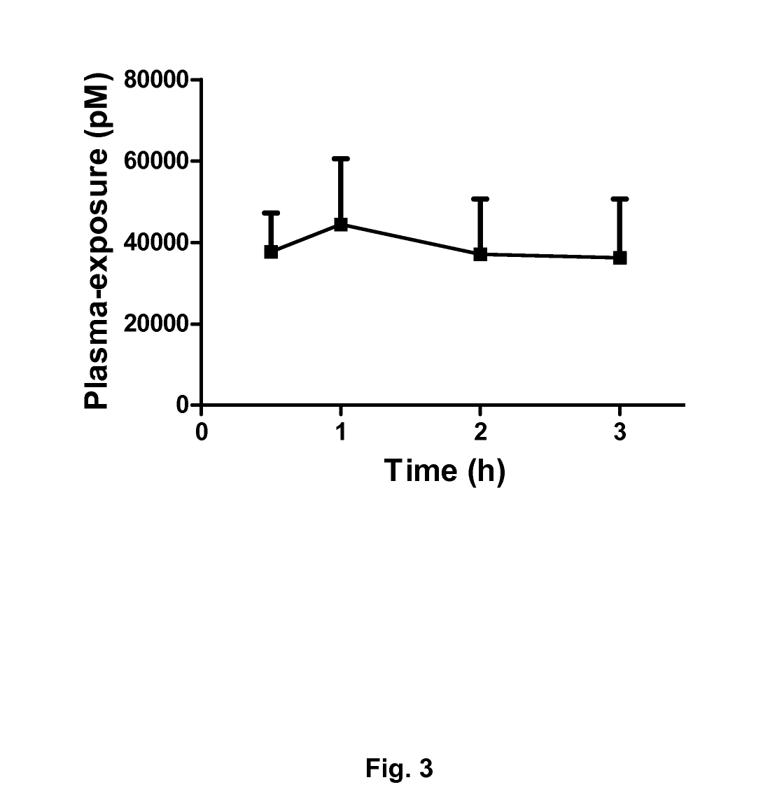

The results are shown in Table 1 (AUC) and FIG. 1-4 show plasma exposure (mean±SEM, in all cases n=6) of semaglutide in rats following gut injection of 100 μl of aqueous formulations of 1000 nmol/ml semaglutide and 55 mg/ml of sodium N-decanoyl leucinate (FIG. 1, squares), sodium N-cocoyl L-glutamate (FIG. 1, diamonds), N-cocoyl glycinate (FIG. 1, triangles), sodium N-myristoyl L-glutamate (FIG. 2, squares), sodium N-dodecanoyl L-glutamate (FIG. 2, triangles), or sodium N-dodecanoyl L-sarcosinate (FIG. 3).

TABLE 1
AUC of semaglutide following administration of aqueous compositions
to rats

| | | Dose corrected |
|---|---|---|
| Permeation enhancer | Carbon atoms | AUC (0-180 min) |
| (55 mg/ml) | in fatty acid | in male SD rats |
| Sodium N-dodecanoyl sarcosinate | C12 | 75 |
| Sodium N-decyl L-leucinate | C10 | 51 |
| Sodium N-cocoyl L-glycinate | Mixture of | 25 |
| | C8/C10/C12 | |
| Sodium N-cocoyl L-glutamate | Mixture of | 7 |
| | C8/C10/C12 | |
| Sodium N-dodecanoyl L-glutamate | C12 | approx. 30 |
| Sodium N-myristoyl L-glutamate | C14 | approx. 250 |

The results in Table 1 demonstrate that fatty acid acylated amino acids are able to provide systemic absorption of semaglutide from the intestines.

### Example 2

### SNEDDS with Fatty Acid Acylated Amino Acid

The purpose of this experiment was to determine oral bioavailability of a GLP-1 peptide using the lipid-based drug delivery system, self-nanoemulsification drug delivery systems (SNEDDS), comprising a fatty acid acylated amino acid. The composition used and the oral bioavailability thereof is shown in Table 2.

TABLE 2
Oral bioavailability of SNEDDS composition with and without fatty acid
acylated amino acid

| | Composition | |
|---|---|---|
| Ingredient | A | B |
| Semaglutide (nmol/mL) | 4012 | 4000 |
| Semaglutide (amount per capsule, mg) | 16.5 | 16.5 |
| Diglycerol caprylate (% w/w) | 60 | 60 |
| Polysorbate 20 (% w/w) | 25 | 30 |
| Sodium N-myristoyl L-glutamate (% w/w) | 4 | 0 |
| Water (% w/w) | 10 | 10 |
| Oral bioavailability of semaglutide | 0.6 ± 0.6% | 0.3 ± 0.5% |

Semaglutide and the fatty acid acylated amino acid were first dissolved in water. Diglycerol octanoate was added and the mixture was stirred at about 300 rpm for 1 hour at room temperature. Then Tween 20 was added and the mixture was stirred at about 300 rpm for 1.5 hour to prepare the final formulation.

The formulation was hand-filled into VegiCaps (1 g into each capsule) and then entero coated in a pan coater with Eudragit L30-D55:NE 30D 50:50 mixture to a weight gain of 8% w/w.

The results showed an oral bioavailability of semaglutide of 0.6±0.6% following oral administration of capsules with SNEDDS containing 4% sodium N-myristoyl L-glutamate to Beagle dogs, hereby showing the relatively high efficacy of this formulation.

### Example 3

### Solid Composition with Fatty Acid Acylated Amino Acid

The purpose of this experiment was to determine oral bioavailability of a GLP-1 peptide using an oral solid dosage form comprising a fatty acid acylated amino acid. Tablets comprising semaglutide and sodium N-myristoyl L-glutamate were prepared by mixing all ingredients and compressing the mixture into tablets.

### Rat Pharmacokinetic Experiment

The rats were taken into general anaesthesia. The abdomen was opened and the intestine was arranged so that the jejunum was exposed. The gut was ligated (to facilitate insertion of catheter) and approx. 1 cm distal there is made a small cut in the intestinal wall with a scissor. A silicone catheter was inserted into the jejunum approximately 50 cm proximal to the cecum measured with scale. Catheters were loaded with tablets and placed without syringe and needle, and 2 ml saline is dosed into abdomen before incision is closed with wound clips.

A metal plunger was introduced into the catheter just prior to dosing, and the tablet was pulled out at the catheter at time t=0 min. Blood samples were collected 30, 60, 120 and 180 min after dosing into EDTA tubes from the tail vein and centrifuged. Plasma was separated to PCR-plates and immediately frozen. Plasma sample were analysed for semaglutide by a LOCI assay.

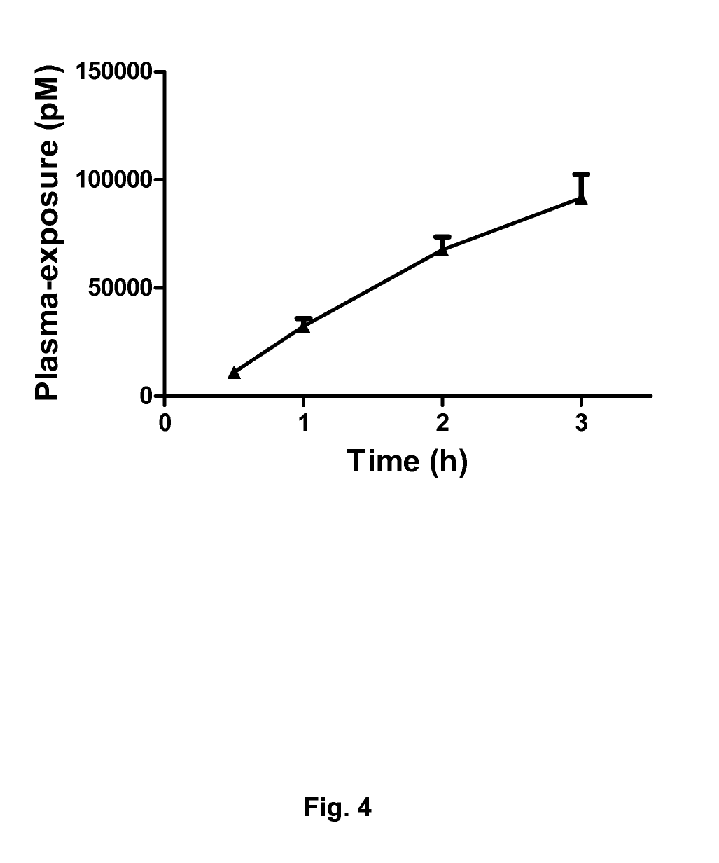

The results are shown in FIG. 4 (plasma exposure mean±SEM, n=8). The results show that plasma exposure of semaglutide following intestinal administration of a solid composition was comparable to the exposure from liquid composition with sodium N-myristoyl L-glutamate. Hence, the permeation enhancing effect of sodium N-myristoyl L-glutamate was maintained in a solid dosage form.

### Example 4

### Transport of Semaglutide Across Caco-2 Cell Monolayers in the Presence of Fatty Acid Acylated Amino Acids (FA-aas)

The purpose of this experiment was to determine the permeation enhancing effect of different fatty acid acylated amino acids on the transepithelial absorption of a GLP-1 peptide in Caco-2 monolayers.

### Cell Culturing

Caco-2 cells were obtained from the American Type Culture Collection (Manassas, Va.). Cells were seeded in culturing flasks and passaged in Dulbecco's Modified Eagle′ medium supplemented with 10% fetal bovine serum, 1% penicillin/streptomycin (100 U/ml and 100 μg/ml, respectively), 1% L-glutamine and 1% nonessential amino acids. Caco-2 cells were seeded onto tissue culture treated polycarbonate filters in 12-well Transwell® plates (1.13 cm2, 0.4 μm pore size) at a density of 10⁵ cells/well. Monolayers were grown in an atmosphere of 5% CO₂-95% O₂ at 37° C. Growth media were replaced every other day. The experiment was performed on day 10-14 after seeding of Caco-2 cells.

### Transepithelial Transport

The amount of compound transported from the donor chamber (apical side) to the receiver chamber (basolateral side) was measured. The transport study was initiated by adding 400 μl solution (100 μM of semaglutide+0.5 mM fatty acid acylated amino acids) and 0.4 μCi/μl [3H]manntiol in transport buffer to the donor chamber and 1000 μl transport buffer to the receiver chamber. The transport buffer consisted of Hank's balanced saline solution containing 10 mM HEPES, 0.1% adjusted to pH 7.4 after addition of compounds. The transport of [³H]mannitol, a marker for paracellular transport, was measured to verify the integrity of the epithelium.

Before the experiment, the Caco-2 cells were equilibrated for 60 min with transport buffer on both sides of the epithelium. Buffer was then removed and the experiment initiated. Donor samples (20 μl) were taken at 0 min and at the end of the experiment. Receiver samples (200 μl) were taken every 15 min. The study was performed in an atmosphere of 5% CO₂-95% O₂ at 37° C. on a shaking plate (30 rpm).

In all samples with semaglutide and mannitol, the concentration was determined using a LOCI assay and scintillation counter, respectively.

Before and during the experiment the transepithelial electrical resistance (TEER) of the cell monolayers was monitored. In selected experiments, the transport buffer were changed to culturing medium after end of experiment and the TEER measured 24 h after experiment. The TEER was measured with EVOM™ Epithelial Voltohmmeter connected to Chopsticks.

The results are shown in Table 3.

TABLE 3
Permeation of semaglutide across Caco-2 monolayers in the presence and
absence of fatty acid acylated amino acids.

| | C-atoms | | |
|---|---|---|---|
| | in | Pₐₚₚ | SD |
| Permeation enhancer | fatty acid | (10⁻⁸ cm/s) | (10⁻⁸ cm/s) |
| None | | 0.65 | 0.33 |
| Sodium N-decanoyl L- | 10 | 0.31 | 0.11 |
| asparaginate | | | |
| Sodium N-decanoyl L-leucinate | 10 | 2.12 | 0.80 |
| Sodium N-dodecanoyl L- | 12 | 2.53 | 0.72 |
| leucinate | | | |
| Sodium N-dodecanoyl L- | 12 | 5.60 | 4.46 |
| phenylalaninate | | | |
| Sodium N-myristoyl L-leucinate | 14 | 63.88 | 5.91 |
| Sodium N-myristoyl L-valinate | 14 | 78.83 | 14.53 |

Pₐₚₚ (apparent permeability);
SD (standard deviation)

The results show that the permeation enhancing effect of fatty acid acylated amino acids is influenced by both amino acid type and fatty acid chain length. Of those tested, the fatty acid acylated amino acids comprising of a 14-carbon chain length (Sodium N-myristoyl L-leucinate and Sodium N-myristoyl L-valinate) exhibited greatest effect.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising i) a GLP-1 peptide and ii) at least one fatty acid amino acid (FA-aa) or a salt of said FA-aa, wherein said FA-aa comprises an amino acid acylated at a free amino group with a fatty acid, wherein said fatty acid comprises an alkyl group consisting of 5 to 19 carbon atom.

2. The pharmaceutical composition according to claim 1, wherein the composition is an oral pharmaceutical composition.

3. The pharmaceutical composition according to claim 1, wherein said
FA-aa has the general formula I: 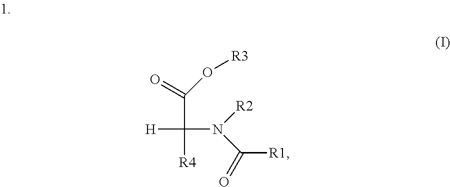
wherein
R1 is an alkyl group consisting of 5 to 19 carbon atoms;
R2 is H (i.e. hydrogen), CH₃ (i.e. methyl group), or covalently attached to R4 via a (CH₂)₃ group;
R3 is H or absent; and
R4 is an amino acid side chain, or covalently attached to R2 via a (CH₂)₃ group.

4. The pharmaceutical composition according to claim 1, wherein R1 is an alkyl group consisting of 7 to 17 carbon atoms.

5. The pharmaceutical composition according to claim 1, wherein R4 is an amino acid side chain selected from the group consisting of a non-cationic amino acid side chain, a non-polar hydrophobic amino acid side chain, a polar non-charged amino acid side chain, or a polar acidic amino acid side chain.

6. The pharmaceutical composition according to claim 1, wherein said FA-aa comprises an amino acid residue selected from the group consisting of a non-cationic amino acid residue, a non-polar hydrophobic amino acid residue, a polar non-charged amino acid residue, or a polar acidic amino acid residue.

7. The pharmaceutical composition according to claim 1, wherein said FA-aa is in the form of its free acid or the salt thereof, such as the sodium salt.

8. The pharmaceutical composition according to claim 1,
wherein the amino acid residue of said FA-aa is selected from the group consisting of sarcosine residue, a glutamic acid residue, and a leucine residue; or
wherein the amino acid residue of said FA-aa is the amino acid residue of an amino acid selected from the group consisting of Alanine (Ala), Valine (Val), Leucine (Leu), Isoleucine (Ile), Phenylalanine (Phe), Tryptophane (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gln), Aspartic acid (Asp), and Glutamic acid (Glu).

9. The pharmaceutical composition according to claim 8, wherein the FA-aa is N-decyl leucine, N-dodecanoyl sarcosine or N-myristoyl glutamine, or a salt thereof.

10. The pharmaceutical composition according to claim 1, wherein the GLP-1 peptide is a GLP-1 analogue or a derivative thereof comprising less than 10 substitutions, deletions or insertions compared to human GLP-1(7-37); and wherein the GLP-1 peptide is optionally an acylated GLP-1 peptide.

11. The pharmaceutical composition according to claim 1, wherein said composition comprises one or more additional pharmaceutically acceptable excipients.

12. The pharmaceutical composition according to claim 1, wherein said composition is in the form of a solid, a liquid, or a semisolid.

13-15. (canceled)

16. A method for treating type 2 diabetes in a subject in need thereof, said method comprising administering to said subject a therapeutically effective amount of the pharmaceutical composition of claim 1.

17. The pharmaceutical composition according to claim 4, wherein R1 is an alkyl group consisting of 9 to 15 carbon atoms.

18. The pharmaceutical composition according to claim 4, wherein R1 is an alkyl group consisting of 11 to 13 carbon atoms.

19. The pharmaceutical composition according to claim 10, wherein the GLP-1 peptide is semaglutide.
